(19) United States
(12) Patent Application Publication
(10) Pub. No.: US 2026/0083329 A1
(43) Pub. Date: Mar. 26, 2026

(54) REAL TIME OPTO-PHYSIOLOGICAL MONITORING METHOD AND SYSTEM

(71) Applicant: Carelight Limited, Altrincham (GB)

(72) Inventors: Sijung Hu, Altrincham (GB); Jiajin Hou, Altrincham (GB); Xiaoyu Zheng, Altrincham (GB); Yasmin Elsahar, Altrincham (GB); Vincent Dwyer, Altrincham (GB)

(21) Appl. No.: 19/111,296

(22) PCT Filed: Sep. 4, 2023
(86) PCT No.: PCT/GB2023/052281

§ 371 Date: Mar. 12, 2025

(30) Foreign Application Priority Data
Sep. 14, 2022 (GB) 2213483.7

(51) Int. Cl. A61B 5/00

(52) U.S. Cl. CPC A61B 5/0059; A61B 5/7203; A61B 5/7267

(57) ABSTRACT

A method of monitoring a subject with an opto-physiological sensor system is described herein. The method comprises obtaining a model of the optical properties of at least one body tissue type to be monitored, wherein the model of the optical properties comprises a definition of static (DC) and dynamic (AC) components of transmitted optical power and a definition of a source-detector separation related to a normalised path length for an illumination source of the opto-physiological sensor: obtaining an indication of at least one physiological property of the subject from a wearable device worn by the subject: obtaining an indication of at least one physical variable from the wearable device worn by the subject; determining, using the opto-physiological model, how the at least one physical variable affects the at least one physiological property; and determining a corrected value for the physiological property based on the determination of how the at least one physical variable affects the at least one physiological property.

### FIELD OF THE INVENTION

The present disclosure relates to a real time opto-physiological monitoring method and system.

### BACKGROUND

Photoplethysmography (PPG) is a non-invasive optical technique for monitoring variation in blood volume or blood flow near the surface of the skin to determine various physiological parameters. The principle uses an illumination source and a photodetector to measure changes in intensity as light is passed through or reflected from body tissue. The detected optical signals are then analysed and correlated with the pulsation of blood through the body, as stimulated by the heartbeat.

Opto-physiological monitoring (beyond but including Beer-Lambert based photoplethysmography, PPG) is applicable to the global mobile health market, which is growing rapidly in developed countries in response to the rising incidence of lifestyle induced chronic diseases and ageing populations. The two main applications of this technology are: i) real time and any-time clinician-monitoring to capture important health parameters of chronically ill subjects or those undergoing post-operative care, and ii) self-monitoring and assessment, for example, for sports performance or to maintain or improve general wellbeing and fitness levels. In addition, monitoring and assessment may be performed to identify or confirm underlying illnesses or to monitor the vital parameters of at-risk subjects to track underlying conditions and provide an early warning signal in order to prevent exacerbation.

In relation to current PPG systems, pulse oximetry devices (which determine blood oxygen saturation) are most common. However, these are often inaccurate as they do not take into account the dynamic changes of tissue optical properties in biological live tissue, motion induced artefacts or the effects of sweat, skin creams or sprays.

In the consumer fitness market, electrical chest-strap based continuous heart rate monitoring systems are often employed. However, these tend to be uncomfortable and obtrusive making them unpopular for frequent use. Other wearable monitoring systems are emerging in the market, including wristwatch-based sensors that can measure individual parameters such as heart rate, heart rate variability, oxygen saturation, and/or respiratory rate. However, no wearable sensor is currently available that can simultaneously measure a wider range of these physiological parameters to meet clinical monitoring standards.

It is therefore an aim of the present invention to provide an opto-physiological (OP) sensor system consisting of 1) one multispectral illumination optical sensor (optical sensor), 2) one bespoke electronics to drive the optical sensor and to capture signals from the optical sensor, 3) one embedded real-time Artificial Intelligence (AI) signal processing algorithm to generate physiological readings, one mobile or web app as a user interface, and 4) one method of monitoring a subject with one optical sensor, which helps to address the aforementioned problems.

WO 2015056007 describes an example of a method of assembling an opto-physiological sensor. WO2021233319A1 describes a premature beat detection method, and an electronic device and medium. WO2021213337A1 describes a usage monitoring method for a wearable electronic device a medium, and the wearable electronic device, relating to the technical field of information processing. US20210393150A1 describes an apparatus for measuring bio-information in a non-invasive manner that includes: a pulse wave sensor configured to measure a plurality of pulse wave signals having different wavelengths from an object; a contact pressure sensor configured to measure contact pressure of the object while the plurality of pulse wave signals are measured; and a processor configured to obtain an oscillometric waveform based on the contact pressure and the plurality of pulse wave signals having the different wavelengths, and obtain bio-information based on the oscillometric waveform. US20220015716A1 describes an electronic device and method for sleep apnoea detection. US20210 330209A1 describes systems, devices, and methods for tracking one or more physiological metrics (e.g., heart rate, blood oxygen saturation, and the like) of a user. US20210236014A1 describes a finger-wearable blood pressure monitor device includes a cuff, a tactile sensor array, and control circuitry. US20210196204A1 describes methods, systems, and method for predicting sensor measurement quality. US20210375473A1 describes a wearable device that can be used for hypertension monitoring. US20210386363A1 describes an electronic device includes a translucent layer that forms a portion of an exterior of the electronic device, an opaque material positioned on the translucent layer that defines micro-perforations, and a processing unit operable to determine information about a user via the translucent layer. US20200297226A1 describes an electronic fitness device that comprises a housing, a first optical transmitter array, a first optical receiver, and a second optical receiver. US20210212620A1 describes a mobile electronic device that is operable to be used in a vehicle and determine a mental state of a user such as drowsiness. US20210353165A1 describes measuring, using a wearable device, a blood pressure of a user includes extracting, using sensor data of the wearable device, features related to a pulse wave; determining a pulse transit time (PTT); scaling at least one of the features using the PTT to obtain a scaled feature; using the scaled feature as an input to a machine-learning (ML) model; and obtaining, using an output of the ML model, the blood pressure of the user. CN111588385A describes an oxyhaemoglobin saturation measuring method and device. US20210121109A1 describes methods and systems for a light-emitting diode (LED) drive circuit of an optical probe. US20210345899A1 describes a multi-sensor patch for simultaneous abdominal monitoring of maternal and foetal physiological data includes a multi-layer flexible substrate. WO2021250224A1 describes an apparatus and method for estimating one or more hemodynamic parameters such as cardiac output or stroke volume. US20220000435A1 describes a method and apparatus for determining respiratory information for a subject. WO2021101705A1 describes devices, systems, and techniques for monitoring a subject condition. US20210290060A1 describes systems and methods are provided for remote subject management and monitoring. WO2021194622A1 describes a method of measuring a physiological characteristic of a user. US20210060343A1 describes systems and methods for managing pain of a subject. US20210178164A1 describes a method and apparatus for pain management with sleep detection.

Klodell C T, et al., Oximetry-derived perfusion index for intraoperative identification of successful thoracic sympathectomy. Ann Thorac Surg 2005; 80:467-70, describes how In patients with hyperhidrosis of the upper extremity, the intraoperative PI derived from pulse oximetry is an additional indicator of successful thoracic sympathectomy.

Park J, Seok H S, Kim S-S and Shin H (2022) Photoplethysmogram Analysis and Applications: An Integrative Review. Front. Physiol. 12:808451, is a review paper that aims to examine existing research on photoplethysmogram concerning its generation mechanisms, measurement principles, clinical applications, noise definition, pre-processing techniques, feature detection techniques, and post-processing techniques for photoplethysmogram processing, especially from an engineering point of view

### SUMMARY OF THE INVENTION

Aspects of the invention are as set out in the independent claims and optional features are set out in the dependent claims. Aspects of the invention may be provided in conjunction with each other and features of one aspect may be applied to other aspects.

In a first aspect there is described a method of monitoring a subject with an opto-physiological sensor system that takes into account physical factors. The opto-physiological sensor system comprises at least one sensor. The method comprises:
obtaining a model of the optical properties of at least one body tissue type to be monitored, wherein the model of the optical properties comprises a definition of static and dynamic components of transmitted optical power and a definition of a source-detector separation related to a normalised path length for an illumination source of the opto-physiological sensor;
obtaining an indication of at least one physiological property of the subject from a wearable device comprising the at least one sensor worn by the subject;
obtaining an indication of at least one physical variable from the wearable device comprising the at least one sensor worn by the subject; and
determining, using the model, how the at least one physical variable affects the at least one physiological property; and
determining a corrected value for the physiological property based on the determination of how the at least one physical variable affects the at least one physiological property.

The at least one sensor may comprise at least one sensor configured to obtain an indication of at least one physiological property and at least one sensor configured to obtain an indication of at least one physical variable or parameter (such as movement). The at least one sensor configured to obtain an indication of at least one physiological property may be an optical sensor. The optical sensor may be a multispectral optical sensor, capable of receiving a range of wavelengths of electromagnetic radiation.

The at least one sensor configured to obtain an indication of at least one physiological property may be configured to obtain an indication of at least one of: heart rate (HR), perfusion index (PI), oxygen saturation (SpO₂%), respiration rate (RR), blood pressure (BP), pulse rate variability (PRV), pulse transmitted time (PTT), pulse wave velocity (PWV). For example, the at least one sensor configured to obtain an indication of at least one physical variable or parameter may be configured to obtain an indication of at least one of contact pressure F_{c}; contact temperature T_{A}; acceleration α_{x,y,z}; angular velocity Ω_{x,y,z}; and absolute orientation º_{x,y,z}.

Obtaining a model of the optical properties of at least one body tissue type to be monitored may comprise obtaining a model of the opto-physiological properties of at least one body tissue type to be monitored. Obtaining a model of the optical properties of at least one body tissue type to be monitored may comprise obtaining a model of the optical properties based on an individual's physiological variable. An individual's physiological variable may comprise at least one of: body tissue type, tissue/skin age, body mass, presence of tattoos, tissue location and/or skin pigmentation to be monitored. In this way the model can be used to accurately determine a corrected value for a physiological property for an individual regardless of their age, skin tone, body mass and presence of tattoos.

Obtaining an indication of at least one physiological property of the subject from a wearable device worn by the subject may comprise obtaining at least one physiological reading of the subject from a wearable device worn by the subject. In such cases determining, using the model, how the at least one physical variable affects the at least one physiological property may comprise determining, using the model, how the at least one physical variable affects the at least one physiological reading.

The definition of a source-detector separation related to a normalised path length for an illumination source of the optical sensor may be a definition of a source-detector separation related to an optimized path length for an illumination source of the optical sensor.

The method may further comprise determining measurements of accurate physiological parameters using the corrected value for the physiological property, the physiological parameters comprising at least one of: heart rate (HR), perfusion index (PI), oxygen saturation (SpO₂%), respiration rate (RR), blood pressure (BP), pulse rate variability (PRV), pulse transmitted time (PTT), pulse wave velocity (PWV).

The at least one physical variable may comprise at least one of: contact pressure F_{c}; contact temperature T_{A}; acceleration α_{x,y,z}; angular velocity Ω_{x,y,z}; and absolute orientation º_{x,y,z}.

The method may further comprise defining infinitesimal optical power dP that is transmitted through to point (x, y) on a surface of the body tissue type as per Equation 1:

$$ρ ( λ , {l}^{′} ( {x}^{′} , {y}^{′} ) ) : dP ( λ , x , y ) = \int \int {I}_{0} ( λ , {x}^{′} , {y}^{′} ) \times {e}^{- ρ ( λ , {l}^{′} ( {x}^{′} , {y}^{′} ) )} {dx}^{′} {dy}^{′} \qquad [ Eq . 1 ]$$

wherein I₀(λ, x, y) represents the total light that enters across the entire surface of the body tissue type and is subject to exponential decay, which is a function of the optical density ρ(λ) of the body tissue type;
wherein a source-detector separation is defined in terms of an illumination source at point (xₛ, yₛ) and an arbitrary detector at point (x, y) on the surface of the body tissue type, such that l′(x, y)=\sqrt{(x−xₛ)²+(y−yₛ)²}; and wherein for a detector of a finite rectangular area on the surface of the body tissue type defined by vectors x and y, spanning from x₋ to x₊ and from y₋ to y₊, the optical power received by the detector is as per Equation 2:

$$P ( λ , x , y ) = {\int }_{{x}_{-}}^{{x}_{+}} {\int }_{{y}_{-}}^{{y}_{+}} d P ( λ , {x}_{d} , {y}_{d} ) d {x}_{d} d {y}_{d} \qquad [ Eq . 2 ]$$

The model may comprise defining an optical response of a dynamic and multi-layered body tissue type in terms of its dynamic optical density ρ(λ, l′,t), using a normalised physiological pulse function ψ(t), and absorption, scattering and pulsatility coefficients μₐᵢ(λ), μₛᵢ(λ) and μₚᵢ(λ) respectively, where a layer number i ranges from 1 to N as per Equation 3:

$$ρ ( λ , {l}^{′} , t ) = {\sum }_{i = 1}^{N} ( {μ}_{a i} ( λ ) \times L ( {μ}_{s i} ( λ ) , {l}^{′} ) \times ( 1 + {μ}_{p i} ( λ ) \times ψ ( t ) ) ) \qquad [ Eq . 3 ]$$

The model may comprise separation of static ρ̅(λ, l′) and dynamic \tilde{ρ}(λ, l′, t) components of Equation 3 as per Equations 4 to 6:

$$ρ ( λ , {l}^{′} , t ) = \overline{ρ} ( λ , {l}^{′} ) + \tilde{ρ} ( λ , {l}^{′} , t ) \qquad [ Eq . 4 ]$$

$$\overline{ρ} ( λ , {l}^{′} ) = {\sum }_{i = 1}^{N} ( {μ}_{a i} ( λ ) \times L ( {μ}_{s i} ( λ ) , {l}^{′} ) ) \qquad [ Eq . 5 ]$$

$$\tilde{ρ} ( λ , {l}^{′} , t ) = {\sum }_{i = 1}^{N} ( {μ}_{a i} ( λ ) \times L ( {μ}_{s i} ( λ ) , {l}^{′} ) \times {μ}_{p i} ( λ ) \times ψ ( t ) ) \qquad [ Eq . 6 ]$$

The model may comprise using a sum rule of integration on Equations 1 and 2 to define static P̅(λ, x, y) and dynamic \tilde{P}(λ, x, y, t) components of transmitted optical power for a rectangular detector defined by vectors x and y as per Equations 7, 8 and 9:

$$P ( λ , x , y , t ) = \overline{P} ( λ , x , y ) + \tilde{P} ( λ , x , y , t ) \qquad [ Eq . 7 ]$$

$$\overline{P} ( λ , x , y ) = {\int }_{{x}_{-}}^{{x}_{+}} {\int }_{{y}_{-}}^{{y}_{+}} ( \int \int {I}_{0} ( λ , {x}^{′} , {y}^{′} ) \times {e}^{- \overline{ρ} ( λ , {l}^{′} ( {x}^{′} , {y}^{′} ) )} {dx}^{′} {dy}^{′} ) d {x}_{d} d {y}_{d} \qquad [ Eq . 8 ]$$

$$\tilde{P} ( λ , x , y , t ) = {\int }_{{x}_{-}}^{{x}_{+}} {\int }_{{y}_{-}}^{{y}_{+}} ( \int \int {I}_{0} ( λ , {x}^{′} , {y}^{′} ) \times {e}^{- \tilde{ρ} ( λ , {l}^{′} ( {x}^{′} , {y}^{′} ) , t )} {dx}^{′} {dy}^{′} ) d {x}_{d} d {y}_{d} \qquad [ Eq . 9 ]$$

The model may comprise defining an optimum source-detector separation l′(λ) for an illumination source at wavelength/as per Equation 10:

$${l}^{′} ( λ ) = {max}_{( x , y )} ( \vert {min}_{t} ( \tilde{P} ( λ , x - x , y - y , t ) ) \vert / \overline{P} ( λ , x - x , y - y ) ) \qquad [ Eq . 10 ]$$

The model may comprise assuming cylindrical symmetry and optical homogeneity of the body tissue type to be monitored such that an optimum source-detector separation l′(λ) is expressed as a circle centred on the position of the illumination source (xₛ, yₛ), or conversely centred on the position of the detector (x_{d}, y_{d}), as per Equation 11.

$${l}^{′} ( λ ) = \sqrt{{( x - {x}_{s} )}^{2} + {( y - {y}_{s} )}^{2}} = \sqrt{{( x - {x}_{d} )}^{2} + {( y - {y}_{d} )}^{2}} \qquad [ Eq . 11 ]$$

In some examples the at least one physical variable comprises at least one of acceleration α_{x,y,z}, angular velocity Ω_{x,y,z} and absolute orientation º_{x,y,z}, and wherein determining, using the model, how the at least one physical variable affects the at least one physiological property comprises modelling the pumping action of the heart to determine a volumetric blood flow rate.

Modelling the pumping action of the heart may comprise determining a pressure gradient in the form of Equation 12

$$\frac{\partial p}{\partial z} = {A}_{0} + {A}_{1} cos {ω}_{p} t \qquad [ Eq . 12 ]$$

where A₀ is the constant component of the blood pressure gradient from the heart, A₁ is the amplitude of the fluctuating component and ω_{ρ}=2πfₚ where fₚ is the pulse frequency, and determining the volumetric blood flow rate Q via equations 13 and 14

$$ρ \frac{\partial {u}_{z}}{\partial t} = ρ {a}_{0} cos ( {ω}_{0} t + ∅ ) + {A}_{0} + {A}_{1} cos {ω}_{p} t + {μ}_{f} ( \frac{{\partial }^{2} {u}_{z}}{\partial {r}^{2}} + \frac{1}{r} \frac{\partial {u}_{z}}{\partial r} ) \qquad [ Eq . 13 ]$$

$$Q = 2 π {\int }_{0}^{R} r \overline{{u}_{z}} dr \qquad [ Eq . 14 ]$$

where ρ and μ_{f} are the density and viscosity respectively of the blood flowing through the blood vessels, and u_{z} is the velocity of the blood flow in the axial direction, and where the z-axis is taken along the axis of the arterial blood segment, and r is taken along the radial direction as the combination of acceleration, angular velocity; and absolute orientation hat can be obtained when obtaining the indication of the at least one physical variable from the wearable device.

In some examples the at least one physical variable comprises temperature, and wherein determining, using the model, how the at least one physical variable affects the at least one physiological property comprises modelling the density of a specific tissue type as a function of wavelength of the illumination source of the opto-physiological sensor and temperature.

Modelling the density of a specific tissue type as a function of wavelength of the illumination source of the opto-physiological sensor system and temperature may comprise modelling the density of the tissue as per equation 15:

$$ρ ( λ , t ) {C}_{p} V \frac{dT}{dt} = {h}_{air} A ( T - {T}_{air} ) + {ρ}_{b} {C}_{{p}_{b}} {ω}_{b} ( t ) ( {T}_{A} - T ) \qquad [ Eq . 15 ]$$

where ρ(λ, t) indicates the density of a specific tissue type, Cₚ is the tissue specific heat, V is the specific tissue volume, and A is the tissue surface area, such that A=πD²/2. V and A are dependent on the average tissue diameter D=2R, where R is the average radius of the tissue surface area A. The density and specific heat of blood are denoted by ρ_{b} and C_{pb} respectively, whereas ωₚ is the volumetric blood flow. T_{A} is the arterial temperature and is treated as a constant or equal to body temperature, whereas hₐᵢᵣ is the heat transfer coefficient at the skin surface as dominated by the environmental heat exchange. Tₐᵢᵣ represents the surroundings temperature.

In some examples, modelling the density of a specific tissue type as a function of wavelength of the illumination source of the opto-physiological sensor and temperature comprises modelling tissue temperature as a result of blood perfusion as defined by thermoregulation for multi-layers with multi-wavelength illuminations as per equation 16:

$${ρ ( λ , t )}_{N} {C}_{p N} \frac{d {T}_{N}}{d t} = {k}_{N} {∇}^{2} {T}_{N} + {ρ}_{b} {C}_{{p}_{b}} {ω}_{b N} ( t ) ( {T}_{A} - {T}_{N} ) + {q}_{m N} \qquad [ Eq . 16 ]$$

where k_{N}, ρ_{N}, C_{pN}, ω_{bN}, and q_{mN} represent the thermal conductivity, density, specific heat, blood perfusion and metabolic heat generation of the respective tissue layer (N), ρ_{b} is the blood density, whereas C_{pb} represents the specific heat of blood and T_{A} is the body temperature. While the body temperature is reached at the balance, e.g., T_{A}−T_{N}=0, T_{N} is treated as a constant. In most circumstances, T_{A}−T_{N}> or <0, thus Eq. 16 represents the change of optical density ρ(λ, t).

In some examples the at least one physical variable comprises contact pressure F_{c} of the opto-physiological sensor with the subject, and wherein determining, using the model, how the at least one physical variable affects the at least one physiological property comprises modelling a change in the optical density ρ(λ, t) of a specific tissue type based on a change in the optical path length l′(λ).

In some examples determining, using the model, how the at least one physical variable affects the at least one physiological property comprises modelling the optical density ρ(λ, t) of a specific tissue type as a function of wavelength (λ) and temperature (T_{A}−T_{N}) as being proportional to the contact force F_{c} when the contact force F_{c} is within a selected range, and modelling the optical density of a specific tissue type as a function of wavelength (λ) and temperature (T_{A}−T_{N}) as being proportional to the inverse of the contact force F_{c} when the contact force F_{c} is outside of the selected range.

In some examples the method may comprise modelling the changes in the optical path length l(λ, t) due to increased or decreased contact force as inducing a change in the optical density ρ(λ, t) in terms of its AC and DC components given by \tilde{ρ} and ρ̅ respectively, such that

$$\{ \begin{matrix}ρ ( λ , t ) ∝ {F}_{c} , & 0.15 < {F}_{c} < 1.5 \\ ρ ( λ , t ) ∝ \frac{1}{{F}_{c}} , & otherwise\end{matrix} . \qquad [ Eq . 17 ]$$

In another aspect there is provided a wearable opto-physiological sensor system for obtaining physiological properties of the wearer and physical variables of the wearer's movements. The opto-physiological sensor system comprises:
an optoelectronic sensor panel comprising a plurality of photodiodes and a plurality of illumination sources, e.g., light emitting diodes, wherein at least a selection of the plurality of illumination sources, e.g., light emitting diodes are configured to emit light at a different wavelength to the other illumination sources, e.g., light emitting diodes;
a plane pressure sensor for obtaining an indication of contact force (F_{c}) between the opto-physiological sensor and the wearer; and
a printed circuit board comprising a micro-electromechanical sensor for obtaining the physical variables, a micro control processor configured to process signals received from the plurality of micro-electromechanical sensors and the plurality of photodiodes, and a wireless communications interface.

The micro-electromechanical sensor may comprise at least two of a gyroscope, an accelerometer, and a digital compass.

The optoelectronic sensor panel may be separated from the plane pressure sensor by a flexible substrate.

The plane pressure sensor may be separated from the printed circuit board by a flexible substrate.

The printed circuit board needs to be flexible.

The optoelectronic sensor panel comprises a plurality of temperature sensors configured to obtain an indication of the wearer's skin through contact with the wearer's skin.

The optoelectronic sensor panel comprises a plurality of temperature sensors equally spaced apart on the optoelectronic sensor panel.

The processor may be configured to transmit raw sensor data or real-time processed data via the wireless communications interface.

The processor is configured to apply a machine learning model to sensor data obtained from the plurality of photodiodes, pressure data obtained from the plane pressure sensor, and the physical variables, to produce modified output data, and to send the modified output data via the wireless communications interface.

The processor is configured not to obtain sensor data from the plurality of photodiodes or the micro-mechanical sensors in the event that the temperature sensor provides an indication of a temperature outside a selected temperature range.

The micro control processor is configured not to obtain sensor data from the plurality of photodiodes or the micro-mechanical sensors in the event that the plane pressure sensor provides an indication of a contact pressure outside a determined contact force (F_{c}) range.

In another aspect there is provided a method of monitoring a subject with an opto-physiological sensor system. Such a method is particularly suitable, for example, when the subject is undertaking high intense exercise. The method comprises:
obtaining a signal from the wearable device worn by the subject comprising a component indicative of at least one physiological property of the subject and a component indicative of at least one physical parameter;
modelling the obtained signal as a Taylor series being a linear combination of the pulsatile signal and a motion term based on the indication of the at least one physical parameter;
obtaining a corrected signal as a projection of the modelled pulsatile signal onto an orthogonal subspace; and
using the corrected signal to determine at least one physiological property of the subject.

The movement of the sensor creates a small time-varying modulation to the received light signal. Although small, it is on the same scale as the pulsatile signal. Thus, the received light signal, to first approximation, in small terms, consists of DC component+a cardiac pulsatile signal+a motion related signal. Any product term of the ‘motion related’ and the ‘cardiac’ contributions can be ignored to first approximation.

The manner in which this ‘motion’ term relates to the values measured by the triaxial accelerometers is likely to depend upon both the elasticity of the tissue layers and the impact of the associated tissue strains on the absorption and scattering parameters; compressing tissue increases the number of absorbers per unit path length travelled. With a roughly linear-elastic tissue model, the frequencies present in the measured accelerometer signals appear in the ‘motion related signal’ (with some amplitude and phase changes). It is these frequencies that are removed by the method claimed. We assume is that this motion related term can be made up of some weighted sum of measured accelerometer signals (aₓ, a_{y} and a_{z}) and their integrated velocity terms (vₓ, v_{y} and v_{z}) with weights N₁ to N₆. The inclusion of the velocity terms allows for some changes in the amplitudes and phases. The values of the Nₖ, k=1 . . . 6, are chosen to remove the most signal power at motion frequencies from the uncorrected signal over some chosen time clip of the measured signal. This is equivalent to projecting onto an orthogonal subspace (the one orthogonal to that spanned by the six signals aₓ, a_{y}, a_{z}, vₓ, v_{y} and v_{z}). Obtaining a corrected pulsatile signal as a projection of the modelled pulsatile signal onto an orthogonal subspace is equivalent to minimising the integral shown in Equation 30 with respect to the undetermined multipliers Nₖ over the selected time interval T_{M} where s (t) is the obtained pulsatile signal and aₖ represents the measured values from the triaxial accelerometer and their integrated velocities

$$f ( N ) = {\int }_{0}^{{T}_{M}} {( s ( t ) - {\sum }_{i = 1}^{6} {N}_{k} {a}_{k} ( t ) )}^{2} d t \qquad [ Eq . 30 ]$$

The corrected signal is then

$$s ( t ) - {\sum }_{k = 1}^{6} {N}_{k} {a}_{k} \qquad [ Eq . 31 ]$$

where the Nₖ values are those that minimise f(N).

The integral may be performed over a rolling time window of length T_{M}. The rolling time window may be, for example, T_{M}=8 seconds and is refreshed every second.

The signal may comprise a pulsatile signal comprising an AC component and a DC component, and wherein obtaining a corrected pulsatile signal comprises obtaining the projection of the measured AC signal onto an orthogonal subspace.

A large DC contribution l_{DC} may arise from light scattered by local tissue, and the AC component l_{AC}(t) may be much smaller and which includes the pulsatile signal (or pulsatile wave or pulsatile waveform) together with other time-dependent effects.

The method may further comprise filtering the pulsatile signal before minimising the integral to remove any DC component, low frequency baseline shift and/or high-frequency noise.

The method may further comprise filtering the signal with a band-pass filter of zero phase prior to obtaining a corrected pulsatile signal.

Obtaining a signal from the wearable device worn by the subject may comprise obtaining a plurality of pulsatile signals from the wearable device, wherein each of the plurality of pulsatile signals are optical signals at discrete wavelengths.

The method may further comprise performing independent component analysis (ICA) or principal component analysis (PCA) on each of the plurality of signals, optionally after the signal is corrected. For example, motion may be removed, from all wavelengths, first and then a principal component analysis performed on what is left. It is likely to be better that way, rather than to do a PCA first, simply because the latter would effectively allow only one set of values Nₖ, whereas the former gives each wavelength its own set of Nₖ. It is likely that the motion related term depends upon wavelength and so appears differently in the overall reflected signal at that wavelength.

Obtaining an indication of at least one physical parameter from the wearable device worn by the subject may comprise receiving a plurality of signals from the wearable device indicative of respective physical parameters, and normalising the received plurality of signals between 0 and 1 to avoid any non-compliance of physical units.

In another aspect there is provided a method of real-time monitoring a subject with an opto-physiological sensor system. The method helps process data quickly and in real time. The method comprises:
obtaining a pulsatile signal from an optical sensor providing an indication of at least one physiological property of the subject from a wearable device worn by the subject;
obtaining an indication of at least one physical variable from the wearable device worn by the subject; and
determining, based on the obtained indication of the at least one physical variable, a dominant source of noise in the pulsatile signal; and
determining, from a selected list of models to apply, a selected noise removal model to apply to the pulsatile signal; and
determining a corrected value for the physiological property using the selected noise removal model.

The selected list of models may comprise:
orthogonal separation;
a model based on acceleration and/or balance;
a model based on temperature; and
a model based on contact pressure.

Determining, based on the obtained indication of the at least one physical variable, a dominant source of noise in the pulsatile signal may comprise determining whether the obtained indication of at least one physical variable exceeds a selected threshold, and wherein
determining, from a selected list of models to apply, a selected noise removal model to apply to the pulsatile signal may comprise selecting the noise removal model based on the determination of whether the obtained indication of at least one physical variable exceeds a selected threshold.

The method may further comprise obtaining a model of the optical properties of at least one body tissue type to be monitored, wherein the model of the optical properties comprises a definition of static and dynamic components of transmitted optical power and a definition of a source-detector separation related to a normalised path length for an illumination source of the opto-physiological sensor system; and
wherein determining a corrected value for the physiological property using the selected noise removal model comprises determining a corrected value for the physiological property using the model of the opto-physiological properties of the at least one body tissue type and the selected noise removal model.

The physical variable comprises at least one of: contact pressure; temperature; acceleration; angular velocity; and absolute orientation.

In another aspect there is provided a method of training a machine learning model for use in monitoring a subject with a wearable device comprising an opto-physiological sensor system. The method comprises:
obtaining a pulsatile signal providing an indication of at least one physiological property of a subject;
obtaining an indication of at least one physical variable as a function of time from the wearable device;
sending data representative of the pulsatile signal and the indication of the physical variable via a wireless interface to a remote computing platform, i.e., workstation or a computing cloud.
training, at the remote computing platform, a machine learning model based on the received data; and
sending the coefficients/parameters, after the training, from the remote computing platform to an embedded machine learning model in the wearable device.

Training, at the remote computing platform, a machine learning model based on the received data, may comprise training a generative adversarial network (GAN) comprising two generators and a discriminator. One of the two generators is configured to learn a noise-resistant mapping from the input noisy pulsatile signal to reference signals, and the other of the two generators is configured to learn noise distribution in the physical variable captured from the wearable device.

In another aspect there is provided a method of training a machine learning model for use in monitoring a subject with a wearable device comprising an opto-physiological sensor system. The method comprises:
obtaining a pulsatile signal providing an indication of at least one physiological property of a subject;
obtaining an indication of at least one physical variable as a function of time from the wearable device;
processing the pulsatile signal and the indication of the at least one physical variable using a machine learning model to provide an output indicative of the physiological property;
sending data representative of the pulsatile signal and the indication of the physical variable via a wireless interface to a remote computing platform;
training, at the remote computing platform, an improved machine learning model based on the received data; and
sending the improved, trained, machine learning model to the wearable device; and
using the improved, trained, machine learning model to process the pulsatile signal and the indication of the at least one physical variable using a machine learning model to provide an output indicative of the physiological property.

Obtaining a pulsatile signal providing an indication of at least one physiological property of a subject and obtaining an indication of at least one physical variable as a function of time from the wearable device may be performed while a user of the wearable device is performing a selected exercise routine, and wherein the machine learning model is trained at the remote computing platform based on the exercise routine.

In another aspect there is provided a wearable opto-physiological sensor system for obtaining physiological properties of the wearer and physical variables of the wearer's movements. The opto-physiological sensor comprises:
an optoelectronic sensor panel comprising a plurality of photodiodes and a plurality of illumination sources, such as light emitting diodes, wherein at least a selection of the plurality of illumination sources, i.e., light emitting diodes are configured to emit light at a different wavelength to the other illumination sources;
a printed circuit board comprising a micro-electromechanical sensor for obtaining the physical variables, a processor configured to process signals received from the plurality of micro-electromechanical sensors and the plurality of photodiodes, and a wireless communications interface;
wherein the plurality of illumination sources are located on intersections between a plurality of concentric circles, wherein each plurality of concentric circles is centred on a respective one of the plurality of photodiodes.

The plurality of illumination sources comprise at least four distinct illumination sources, each configured to emit light at a distinct wavelength. Four or more wavelength illumination sources give a better option as two wavelength illuminations are requested to work out oxygen saturation (SpO₂%), and the responses of the plurality of photodiodes from different wavelength illuminations may be taken account for cross correlation of each wavelength illumination, to determine the best pulsatile signal(s) obtaining from the device.

The plurality of illumination sources comprises at least sixteen distinct illumination sources each configured to emit light at one of four or more distinct wavelengths, wherein the at least sixteen distinct illumination sources are grouped into groups of four each configured to emit light at one of the four or more distinct wavelengths.

In some examples illumination sources of the shortest wavelength are located at the intersection of the inner circles of the plurality of concentric circles, and wherein illumination sources of a longer wavelength are located at the intersection of concentric circles further out from their centre, such that illumination sources of a longer wavelength are located further away from each of the photodiodes and the wavelength of the illumination sources increases with radial distance from each photodiode.

In some examples the optoelectronic sensor panel comprises one, two and three photodiodes.

At least one of the wavelengths of the illumination sources is at 465 nm

The opto-physiological sensor further comprises at least a plane pressure sensor for obtaining an indication of contact force (F_{c}) between the opto-physiological sensor and the wearer.

In another aspect there is provided a method of obtaining an oxygen saturation (SpO2%) biomarker of a subject, the method comprising:
illuminating a subject with at least four discrete wavelengths, λ₁ λ₂, λ₃ λ₄, to obtain a pulsatile signal at each of the four distinct wavelengths λ₁ λ₂, λ₃ λ₄;
obtaining a ratio r(λ), of the height of the pulsatile signal to the associated DC background level, for the four distinct wavelengths and using these values in pairs to obtain the oxygen saturation biomarker of the subject.
with wavelengths chosen to fulfil the condition that for some A, B, C, D

$$ρ = A σ ( {λ}_{1} , {λ}_{2} ) + B = C σ ( {λ}_{3} , {λ}_{4} ) + D \qquad [ Eq . 32 ]$$

where (λ₁,λ₂)=r(λ₁)/r(λ₂); a condition well met with the present device for chosen wavelength pairings and one which would be implied by most current oxygen saturation measures.

Obtaining a ratio r(λ), of the height of the pulsatile signal to the associated DC background level, for the four distinct wavelengths may be obtained with a wearable opto-physiological sensor as described above, and/or with other sensors, for example a camera.

Obtaining the value of ρ from equation 32 allows the oxygen saturation biomarker to be fitted to a universal (wavelength independent) curve

$${SpO}_{2} = {SpO}_{2} ( ρ ) = α + β ρ + γ {ρ}^{2} , \qquad [ Eq . 33 ]$$

and during which the satisfaction of equation 32 becomes an important step in validation of this biomarker.

In another aspect there is a computer readable non-transitory storage medium comprising a program for a computer configured to cause a processor to perform the method of any of the aspects described above.

### DRAWINGS

Embodiments of the disclosure will now be described, by way of example only, with reference to the accompanying drawings, in which:

FIG. 1 shows an example of an opto-physiological sensor system;

FIG. 2 shows another example of an opto-physiological sensor system;

FIG. 3 shows another example of an opto-physiological sensor system;

FIG. 4 shows a functional schematic view of the optoelectronic hardware system 400; and

FIG. 5 shows a functional schematic view of an example method of processing signals obtained from an optoelectronic sensor such as the optoelectronic system of FIGS. 1 to 3 or the optoelectronic hardware system of FIG. 4.

FIG. 6 shows pulsatile waveforms for a subject with skin type I (Fitzpatrick scale) collected from the back of the left wrist using the opto-physiological monitoring system.

FIG. 7 shows pulsatile waveforms for a subject with skin type II (Fitzpatrick scale) collected from the back of the left wrist using the opto-physiological monitoring system.

FIG. 8 shows pulsatile waveforms for a subject with skin type III (Fitzpatrick scale) collected from the back of the left wrist using the opto-physiological monitoring system.

FIG. 9 shows pulsatile waveforms for a subject with skin type IV (Fitzpatrick scale) collected from the back of the left wrist using the opto-physiological monitoring system.

FIG. 10 shows pulsatile waveforms for a subject with skin type V (Fitzpatrick scale) collected from the back of the left wrist using the opto-physiological monitoring system.

FIG. 11 shows pulsatile waveforms for a subject with skin type VI (Fitzpatrick scale) collected from the back of the left wrist using the opto-physiological monitoring system.

FIG. 12 shows pulsative waveforms for a male subject skin type VI (Fitzpatrick scale) with 4 wavelength opto-physiological monitoring.

FIG. 13 shows pulsative waveforms of male subject skin type VI (Fitzpatrick scale) with 4 wavelength opto-physiological monitoring.

### SPECIFIC DESCRIPTION

The following symbols will be used throughout the description:
μₒ coefficient of absorption
μₛ coefficient of scattering
n refractive index
ρ(λ) optical density
l path length l(λ) light intensity
ρ optical power
μ_{f} blood viscosity
u_{z} blood velocity
Q volumetric blood flow rate
Cₚ tissue specific heat
V specific tissue volume
A tissue surface area
T_{A} arterial temperature
hₐᵢᵣ heat transfer coefficient
Tₐᵢᵣ Surrounding air temperature
k thermal conductivity
qₘ metabolic heat generation
g anisotropy factor
F_{c} contact force
ϵ extinction coefficient
C absorber concentration
Ĥ proxy parameter
T_{M} measurement time interval
a_{x,y,z} acceleration
Ω_{x,y,z} angular velocity º_{x,y,z} absolute orientation

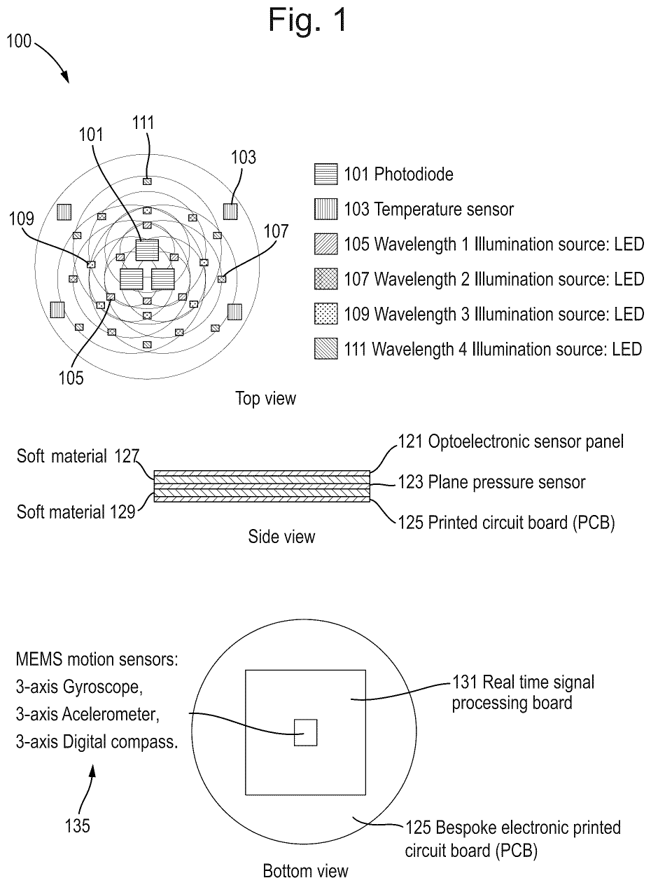

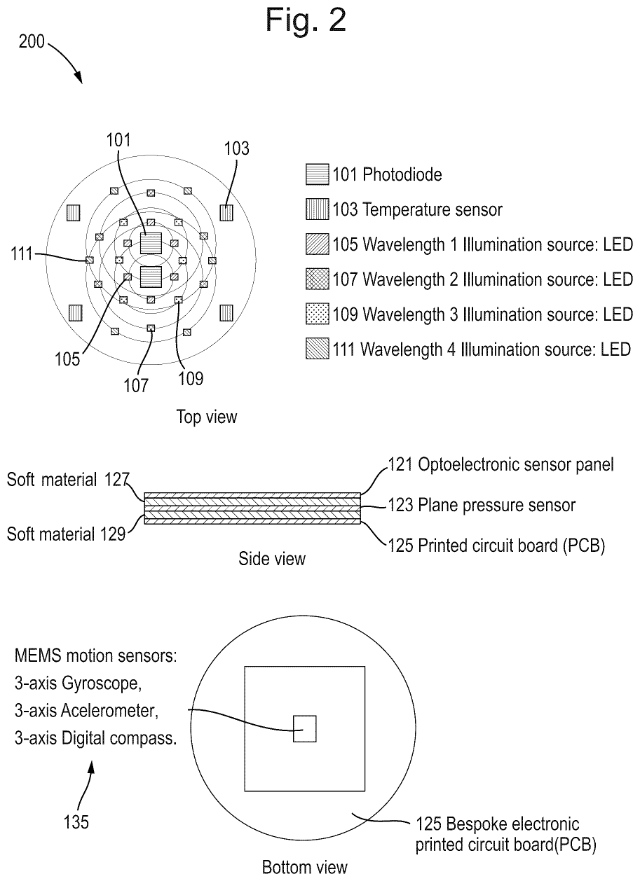

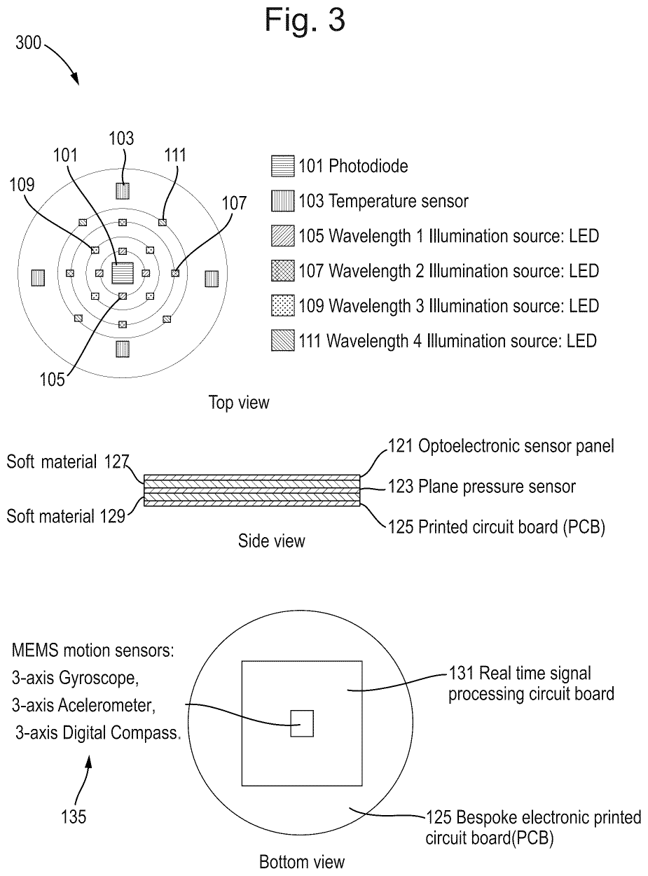

FIG. 1 shows an example opto-physiological sensor system 100 for use in monitoring or determining the opto-physiological properties of a subject. The opto-physiological sensor system 100 may be designed as a wearable, for example as a patch, a watch or finger ring. FIG. 2 shows another example of an opto-physiological sensor system 200, and FIG. 3 shows another example of an opto-physiological sensor system 300.

As can be seen in FIG. 1, the opto-physiological sensor system 100 may comprise one or more sensors (including for example a multi-spectral/wavelength sensor), electronics for controlling and receiving signals from the sensors, and a computer program running on a processor comprising an algorithm configured to process the signals, for example according to an opto-physiological model as is described in more detail below. For example, the opto-physiological sensor system 100 may comprise a photodiode 101, a temperature sensor 103 and a plurality of illumination sources 105, 107, 109, 111, each configured to emit light at a different respective wavelength. It may also comprise MEMS motion sensors 135.

The opto-physiological sensor system 100 is configured to obtain at least one physiological reading of the subject which may provide an indication of at least one physiological property of the subject. The opto-physiological sensor system 100 may also be configured to obtain an indication of at least one physical variable relating to the wearer's movements.

These movements can then be taken into account in an opto-physiological model to obtain more accurate opto-physiological measurements, such as heart rate (HR), perfusion index (PI) oxygen saturation (SpO₂%), respiration rate (RR), blood pressure (BP), pulse rate variability (PRV), pulse transmitted time (PTT), and pulse wave velocity (PWV)

In the example shown in FIG. 1, the opto-physiological sensor system 100 comprises an optoelectronic sensor panel 121 comprising three photodiodes 101, although the example shown in FIG. 2 comprises two photodiodes 102 and the example shown in FIG. 2 only comprises one photodiode 102, and a plurality of illumination sources 105-111 (in the example of FIG. 1, there are 24, in the example of FIG. 2 there are 24, and in the example of FIG. 2 there are 16), which in this example are illumination sources, and optionally at least one temperature sensor 103 (in the examples of FIGS. 1 to 3 there are four).

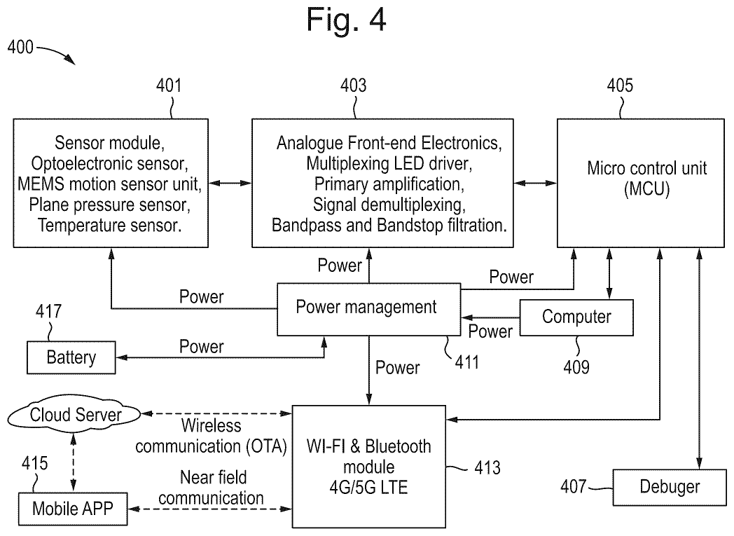

The opto-physiological sensor system 100 also comprises a plane pressure sensor 123 for obtaining an indication of contact force (F_{c}) between the opto-physiological sensor and the wearer, and a flexible printed circuit board 125 comprising a micro-electromechanical sensor 135 for obtaining the physical variables, a micro control processor 405 configured to process signals received from the plurality of micro-electromechanical sensors and the plurality of photodiodes 101. In some examples the opto-physiological sensor system 100 may also comprise an optional wireless communications interface 413 for example located on the printed circuit board 125, as described below with reference to FIG. 4. The micro-electromechanical sensor and the micro control processor may be located on a real time signal processing circuit board 131 mounted on the printed circuit board 125. The real time signal processing circuit board 131 may comprise a computer program and/or instructions that mean it is configured to receive and process sensor signals according to an opto-physiological model, as is described in more detail below.

In this way, the micro-electromechanical sensor 135, 401 and the micro control processor are located on an opposite side of the optoelectronic sensor to the photodiodes, temperature sensor(s) and illumination sources.

In the example shown, the micro-electromechanical sensor 135, 401 comprises a 3-axis gyroscope, a 3-axis accelerometer, and a 3-axis digital compass. As can be seen in FIG. 1, the optoelectronic sensor panel 121 is separated from the plane pressure sensor 123 by a first flexible substrate 127, and the plane pressure sensor 123 is separated from the printed circuit board 125 by a second flexible substrate 129.

The optoelectronic sensor panel 121 comprises a plurality of temperature sensors 103 (in this example, four) configured to obtain an indication of the wearer's skin through contact with the wearer's skin. The plurality of temperature sensors 103 are equally spaced apart on the optoelectronic sensor panel 121.

At least a selection of the plurality of illumination sources 105-111 are configured to emit light at a different wavelength to the other illumination sources. The wavelengths may, for example, be 525 nm, 595 nm, 650 nm, 910 nm, or others. As discussed in more detail below, using a plurality (in this example, four) of different wavelengths may better accommodate different tissue type, tissue location and skin pigmentation. In some examples a wavelength of 475 nm may be preferable.

In the examples of FIGS. 1 and 2, there are six light emitting diodes of each wavelength, and there are four or more different wavelengths, meaning there are 24 or more light emitting diodes in total. As shown in FIGS. 1 and 2, the light emitting diodes 105-111 are located on intersections between a plurality of concentric circles, wherein each plurality of concentric circles is centred on a respective one of the plurality of photodiodes 101. In the example of FIG. 3 where there is only one photodiode, the light emitting diodes 105-111 are equally spaced around the circumference of a series of concentric circles centred on the photodiode 101, however in a manner such that light emitting diodes of an adjacent circle are offset by 90° to increase the separation between illumination sources, e.g., light emitting diodes of adjacent circles.

In the examples of FIGS. 1 and 2, light emitting diodes of the shortest wavelength (wavelength 1) are located at the intersection of the inner circles of the plurality of concentric circles, and illumination sources, e.g., light emitting diodes of a longer wavelength (wavelengths 2 to 4, with 4 being the longest wavelength and 1 being the shortest) are located at the intersection of concentric circles further out from their centre, such that illumination sources, e.g., light emitting diodes 105-111 of a longer wavelength are located further away from each of the photodiodes and the wavelength of the illumination sources, e.g., light emitting diodes increases with radial distance from each photodiode 101. In this way, in the example of FIGS. 1 and 2, the light emitting diodes of wavelength 1 are on the inside, followed by light emitting diodes of wavelength 2 and then 3 and then finally light emitting diodes of wavelength 4 are on the outside. In the example of FIG. 3, the illumination sources, e.g., light emitting diodes are simply arranged such that light emitting diodes of a longer wavelength are located further away from the photodiode 101 and the wavelength of the illumination sources, e.g., light emitting diodes increases with radial distance from the photodiode 101. Preferably at least one of the wavelengths is blue light, for example at 465 nm as this has surprisingly been found to be particularly effective for use in determining SpO₂ for specific tissue type and location.

The micro control processor (MCU) 405 is configured to process the sensor data obtained from the plurality of photodiodes 101, pressure data obtained from the plane pressure sensor 123, and the physical variables from the MEMS motion sensors 135, by applying an opto-physiological model to the data (as described in more detail below), to obtain an indication of a physiological property of a subject/wearer of the optoelectronic sensor 100.

In some examples, the micro control processor 405 may be configured to do this by transmitting raw sensor data or real-time processed data via the wireless communications interface. The micro control processor 405 may be configured to apply a machine learning model to sensor data obtained from the plurality of photodiodes 101, pressure data obtained from the plane pressure sensor 123, and the physical variables, to produce modified output data, and to send the modified output data via the wireless communications interface 413.

In some examples, the micro control processor 405 is configured not to obtain sensor data from the plurality of photodiodes 101 or the micro-mechanical sensors 135 in the event that the temperature sensor(s) 103 provides an indication of a temperature (T) outside a selected temperature range of 18-37° C. on the contacted skin/organ tissue of subject. Additionally, or alternatively, in some examples the micro control processor 405 is configured not to obtain sensor data from the plurality of photodiodes or the micro-mechanical sensors in the event that the plane pressure sensor 123 provides an indication of a contact pressure outside a determined pressure range.

While the wavelength 1 LEDs are fully illuminated, the photodiode(s) start to capture the backlight from the contact skin tissue then switch on the wavelength 2 LEDs to capture the backlight again as the same previous procedure. The wavelength 3, and 4 LEDs perform the same procedures as described. One complete duty cycle of backlight acquisition for four wavelength illumination sources, i.e., LEDs plus a background signal captured by the same photodiode(s), is set as one (1) sampling point consisting of 5 individual and numerical values. Thus, the effect of simultaneous sampling of multiple wavelengths is achieved as anticipated. There are five (5) channel signals of four (4) wavelength illuminations plus one (1) background captured by the photodiode(s) of the multi-wavelength illumination optoelectronic sensor.

As discussed above, in order to process the data received by the optoelectronic sensor 100 and to obtain useful physiological parameters of the subject, an opto-physiological model is applied. In prior art systems, a Beer-Lambert model has been applied to relate the absorption of light to the properties of the material through which the light is travelling. However, it has been found that such an approach is too basic to be truly representative when trying to establish the opto-physiological properties of body tissue and therefore an opto-physiological model, which takes into account various properties of the body tissue to be monitored, is required.

In the present case, the opto-physiological model comprises a definition of static and dynamic components of transmitted optical power and a definition of a source-detector separation related to a normalised path length for an illumination source (e.g., light emitting diode) of the opto-physiological sensor.

The at least one physical variable comprises at least one of: contact pressure; temperature; acceleration; angular velocity; and absolute orientation

In the model, light transmission through a tissue medium can be expressed as an exponential decay as per Equation 1.

$$I ( λ ) = {I}_{0} ( λ ) \times {e}^{- ρ ( λ )} \qquad [ Eq . 1 ]$$

This describes the exponential decay of the intensity of light at wavelength λ entering a medium l₀(λ) as it travels through the tissue medium with an optical density p(A).

The model comprises defining infinitesimal optical power dP that is transmitted through to point (x, y) on a surface of the body tissue type as per Equation 2:

$$ρ ( λ , {l}^{′} ( {x}^{′} , {y}^{′} ) ) : dP ( λ , x , y ) = \int \int {I}_{0} ( λ , {x}^{′} , {y}^{′} ) \times {e}^{- ρ ( λ , {l}^{′} ( {x}^{′} , {y}^{′} ) )} {dx}^{′} {dy}^{′} \qquad [ Eq . 2 ]$$

wherein l₀(λ, x, y) represents the total light that enters across the entire surface of the body tissue type and is subject to exponential decay, which is a function of the optical density ρ(λ) of the body tissue type;
wherein a source-detector separation is defined in terms of a light source at point (xₛ, yₛ) and an arbitrary detector at point (x, y) on the surface of the body tissue type, such that the path length l′(x, y)=\sqrt{(x−xₛ)²+(y−yₛ)²}; and
wherein for a detector of a finite rectangular area on the surface of the body tissue type defined by vectors x and y, spanning from x₋ to x₊ and from y₋ to y₊, the optical power received by the detector is as per Equation 3:

$$P ( λ , x , y ) = {\int }_{{x}_{-}}^{{x}_{+}} {\int }_{{y}_{-}}^{{y}_{+}} d P ( λ , {x}_{d} , {y}_{d} ) d {x}_{d} d {y}_{d} \qquad [ Eq . 3 ]$$

Thus, the model comprises defining an optical response of a dynamic and multi-layered body tissue type in terms of its dynamic optical density ρ(λ, l′, t), using a normalised physiological pulse function ψ(t), and absorption, scattering and pulsatility coefficients μₐᵢ(λ), μₛᵢ(λ) and μₚᵢ(λ) respectively, where a layer number i ranges from 1 to N as per Equation 4:

$$ρ ( λ , {l}^{′} , t ) = {\sum }_{i = 1}^{N} ( {μ}_{a i} ( λ ) \times L ( {μ}_{s i} ( λ ) , {l}^{′} ) \times ( 1 + {μ}_{p i} ( λ ) \times ψ ( t ) ) ) \qquad [ Eq . 4 ]$$

The model comprises a separation of static ρ̅(λ, l′) and dynamic \tilde{ρ}(λ, l′, t) components as DC and AC components separately of Equation 4 as per Equations 5 to 6:

$$ρ ( λ , {l}^{′} , t ) = \overline{ρ} ( λ , {l}^{′} ) + \tilde{ρ} ( λ , {l}^{′} , t ) \qquad [ Eq . 4 ]$$

$$\overline{ρ} ( λ , {l}^{′} ) = {\sum }_{i = 1}^{N} ( {μ}_{a i} ( λ ) \times L ( {μ}_{s i} ( λ ) , {l}^{′} ) ) \qquad [ Eq . 5 ]$$

$$\tilde{ρ} ( λ , {l}^{′} , t ) = {\sum }_{i = 1}^{N} ( {μ}_{a i} ( λ ) \times L ( {μ}_{s i} ( λ ) , {xl}^{′} ) \times {μ}_{p i} ( λ ) \times ψ ( t ) ) \qquad [ Eq . 6 ]$$

The model comprises using a sum rule of integration on Equations 2 and 3 to define static P̅(λ, x, y) and dynamic \tilde{P}(λ, x, y, t) components of transmitted optical power for a rectangular detector defined by vectors x and y as per Equations 7, 8 and 9:

$$P ( λ , x , y , t ) = \overline{P} ( λ , x , y ) + \tilde{P} ( λ , x , y , t ) \qquad [ Eq . 7 ]$$

$$\overline{P} ( λ , x , y ) = {\int }_{{x}_{-}}^{{x}_{+}} {\int }_{{y}_{-}}^{{y}_{+}} ( \int \int {I}_{0} ( λ , {x}^{′} , {y}^{′} ) \times {e}^{- \overline{ρ} ( λ , {l}^{′} ( {x}^{′} , {y}^{′} ) )} {dx}^{′} {dy}^{′} ) d {x}_{d} d {y}_{d} \qquad [ Eq . 8 ]$$

$$\tilde{P} ( λ , x , y , t ) = {\int }_{{x}_{-}}^{{x}_{+}} {\int }_{{y}_{-}}^{{y}_{+}} ( \int \int {I}_{0} ( λ , {x}^{′} , {y}^{′} ) \times {e}^{- \tilde{ρ} ( λ , {l}^{′} ( {x}^{′} , {y}^{′} ) , t )} {dx}^{′} {dy}^{′} ) d {x}_{d} d {y}_{d} \qquad [ Eq . 9 ]$$

The model comprises defining an optimum source-detector separation related to the normalised path length l′(λ) for a light source at wavelength λ as per Equation 10:

$${l}^{′} ( λ ) = {max}_{( x , y )} ( \vert {min}_{t} ( \tilde{P} ( λ , x - x , y - y , t ) ) \vert / P ( λ , x - x , y - y ) ) \qquad [ Eq . 10 ]$$

The model comprises assuming cylindrical symmetry and optical homogeneity of the body tissue type to be monitored such that an optimum source-detector separation related to the normalised path length l′(λ) is expressed as a circle centred on the position of the light source (xₛ, yₛ), or conversely centred on the position of the detector (x_{d}, y_{d}), as per Equation 11.

$${l}^{′} ( λ ) = \sqrt{{( x - {x}_{s} )}^{2} + {( y - {y}_{s} )}^{2}} = \sqrt{{( x - {x}_{d} )}^{2} + {( y - {y}_{d} )}^{2}} \qquad [ Eq . 11 ]$$

The tissue optic properties as stated in the beginning are also governed or affected by acceleration (m²/s), angular velocity (°/s), thermoregulation (° C.), and contact force (N/m²) during movement of physical intensity (PI).

The pumping action of the heart can be represented by the pressure gradient δp/δz as taken in the form as Equation 12:

$$\frac{\partial p}{\partial z} = {A}_{0} + {A}_{1} cos {ω}_{p} t \qquad [ Eq . 12 ]$$

where A₀ is the constant component of the blood pressure gradient from the heart, A₁ is the amplitude of the fluctuating component as in the form of systolic and diastolic blood pressures), ωₚ=2πfₚ and fₚ as the pulse frequency.

Here the artery blood wall is treated as rigid, the blood flow is laminar, there is rotational symmetry of flow, and the variation of velocity along the blood vessel length is small as compared to the rate of change of velocity with respect to time, then in blood vessel, i.e., artery as cylindrical, polar coordinates (r, Ø, z):

$$ρ \frac{\partial {u}_{z}}{\partial t} = ρ {a}_{0} cos ( {ω}_{0} t + ∅ ) + {A}_{0} + {A}_{1} cos {ω}_{p} t + {μ}_{f} ( \frac{{\partial }^{2} {u}_{z}}{\partial {r}^{2}} + \frac{1}{r} \frac{\partial {u}_{z}}{\partial r} ) \qquad [ Eq . 13 ]$$

where ρ and μ_{f} are the density and viscosity respectively of the blood flowing through the blood vessels, and u_{z} is the velocity of the blood flow in the axial direction (angular). Thus, these two physical states of blood flow, e.g., laminar and angle direction, interpret the governors of acceleration and balance of blood flow in physical movement.

The volumetric blood flow rate Q (in the transformed space) can be determined as:

$$Q = 2 π {\int }_{0}^{R} r \overline{{u}_{z}} d r \qquad [ Eq . 14 ]$$

where the z-axis is taken along the axis of the arterial blood segment, and r is taken along the radial direction as the combination of acceleration and balance angular that can be determined and captured by a 3-axis accelerometer and 3-axis gyroscope.

Q can be also spread into capillary or peripheral blood vessels as crossly connected with the tissue body in the combination of the same mechanism of Laminar flow and balance angular. These dynamic systolic and diastolic behaviours can be reflected on changes of tissue optic properties, e.g., a coefficient of absorption (μₐ), a coefficient of scattering (μₛ), a refractive index (n), an optical density ρ(Δ).

Since capillary or peripheral blood flow is governed by major or branched arterial blood vessels, the contents and density of these capillary or peripheral blood are varied with the changes of blood flow from local or even whole cardiac system. These phenomena can be captured or detected by multi-wavelength illumination optoelectronic sensor as truly reflected by the dynamic systolic and diastolic behaviours and the amplitude changes corresponding in differences with different wavelength illuminations.

Tissue temperature (° C.) is a direct result of blood perfusion as defined as thermoregulation, that reflects the changes from tissue composition, skin thickness, surface area, tissue volume, and ambient temperature in presence of live tissue nature or body tissue surroundings.

For a specific tissue type, the phenomenon can be expressed as Eq 15:

$$ρ ( λ , t ) {C}_{p} V \frac{d T}{d t} = {h}_{a i r} A ( T - {T}_{a i r} ) + {ρ}_{b} {C}_{{p}_{b}} {ω}_{b} ( t ) ( {T}_{A} - T ) \qquad [ Eq . 15 ]$$

where ρ(λ, t) indicates the density of a specific tissue type, Cₚ is the tissue specific heat, V is the specific tissue volume, and A is the tissue surface area, such that A=πD²/2.

V and A are dependent on the average tissue diameter D=2R, where R is the average radius of the tissue surface area A. The density and specific heat of blood are denoted by ρ_{b} and C_{pb} respectively, whereas ω_{b} is the volumetric blood flow. T_{A} is the arterial temperature and is treated as a constant or equal to body temperature, whereas hₐᵢᵣ is the heat transfer coefficient at the skin surface as dominated by the environmental heat exchange. Tₐᵢᵣ represents the surroundings temperature.

For multi-layers with multi-wavelength illuminations:

$${ρ ( λ , t )}_{N} {C}_{p N} \frac{d {T}_{N}}{d t} = {k}_{N} {∇}^{2} {T}_{N} + {ρ}_{b} {C}_{{p}_{b}} {ω}_{b N} ( t ) ( {T}_{A} - {T}_{N} ) + {q}_{m N} \qquad [ Eq . 16 ]$$

where k_{N}, ρ_{N}, C_{pN}, ω_{bN}, and q_{mN} represent the thermal conductivity, density, specific heat, blood perfusion and metabolic heat generation of the respective tissue layer (N). In this equation, ρ_{b} is the blood density, whereas C_{pb} represents the specific heat of blood. T_{A} is the body temperature and is treated as a constant.

The thermoregulation presents body temperature variation governed by not only major or most of arterial blood flow but also extended to local tissue temperature changes. In either way, the local tissue temperature changes could simulate the thermoregulation to impact on cardiac blood flow reflecting body temperature variation. In the reality of opto-physiological monitoring, the amplitude of pulsatile waveform or pulse wave can be detected thus clearly indicating the variations of these tissue optic properties of the coefficient of absorption (μₐ), coefficient of scattering (μₛ), refractive index (n), and optical density ρ(Δ, t).

The contact force (F_{c}) as the contact pressure is a physical variable or factor that affects the generated signal's amplitude and in turn, it influences the detected signal-to-noise ratio. Opto-physiological monitoring shows that contact pressure that is either excessive or insufficient induces variations in the detected pulsatile waveforms or pulse waves. With increasing contact force (F_{c}) between the sensor and the skin, the DC amplitude increases, whereas the AC component increases first. High contact force (F_{c}) between opto-physiological sensors and the human skin shows an increase in a signal's AC and DC components, up to a maximum range (specified as the optimal contact pressure). With increased pressure beyond this range, the pulsatile waveform is no longer recognisable.

With reference to [Eq.4-6], the changes in the optical path length due to increased or decreased contact force induce a change in the optical density ρ(λ, t) in terms of its AC and DC components given by \tilde{p} and p̅ respectively, such that

$$\{ \begin{matrix}ρ ( λ , t ) ∝ {F}_{c} , & 0.15 < {F}_{c} < 1.5 \\ ρ ( λ , t ) ∝ \frac{1}{{F}_{c}} , & otherwise\end{matrix} . \qquad [ Eq . 17 ]$$

Pulse amplitudes peak at different contact forces, specifying that individual differences induce changes in relation to the actual force exerted at the artery walls. The range for the peak pulse amplitude is between, for example, 0.15 N to 1.5 N. The sensor system picks up the contact force signal and processes these signals individually with AI driving training strategy.

The optical properties of the tissues, which are wavelength dependent, vary in accordance with the contact pressure values. The deformation of the tissues' structures due to contact pressure affects the scattering properties of the tissue, in particular the scattering phase and the anisotropy factor (g).

FIG. 4 shows a functional schematic view of an example opto-physiological sensor system, which may be an optoelectronic hardware system, 400. The opto-physiological sensor system 400 shown in FIG. 4 comprises a sensor module 401 coupled to a micro control unit (MCU) 405 via an analogue front end 403. The analogue front end 403 comprises a multiplexing LED drive, an amplifier, a band-pass filter, a band-stop filter and a demultiplexer. It will be understood that the features of the opto-physiological sensor system of FIGS. 1 to 3 may be employed with the opto-physiological sensor system 400 of FIG. 4, and vice-versa. For example, the sensor module 401 may comprise the photodiode 101, a temperature sensor 103 and plurality of illumination sources 105, 107, 109, 111, as well as the MEMS motion sensors 135 of FIGS. 1 to 3.

The sensor module 401, analogue front end 403 and microcontroller 405 are coupled to a power management module 411 which is in turn coupled to a battery 417. The micro control unit 405 is also coupled to an optional debugger 407 and an optional computer 409. A wireless communications interface 413 is coupled in parallel to the power management module 411 and the micro control unit 405. The wireless communications interface 413 is configured to communicate with a cloud server and a mobile app 415.

The sensor module 401 comprises:
1. multi-wavelength illumination optoelectronic sensor (named optoelectronic sensor),
2. MEMS motion sensor with one 3-axis gyroscope, one 3-axis accelerometer, and one 3-axis digital compass,
3. One plane pressure sensor, and
4. Four temperature sensors.

The signals captured by the sensor module 401 are divided into two parts.
1. The first part is that 5-channel signals captured by the photodiode(s) are amplified by the primary stage of the Analogue Front-end Electronics (AFE) 403, and then passed to the MCU 405 after being filtered by a combination of a band-pass filter and a band-stop filter.
2. The second part is the reference signals captured by the plane pressure sensor, four (4) temperature sensors, the MEMS sensor unit. These reference signals are directly transmitted to the MCU 405 after a primary amplification.

The MCU 405 plays four roles:
1. Analogue to digital conversion (ADC),
2. Control and driver of AFE 403,
3. Real-time signal processing with embedding AI algorithms, and
4. Real-time data/readings transmission via the wireless communications interface 413 of WI-FI® & Bluetooth®, 4G/5G LTE.

The MCU 405 may therefore comprise a computer program and/or instructions that mean it is configured to receive and process sensor signals according to an opto-physiological model, as described above.

These processed or unprocessed signals can be transmitted to the mobile app 405 through WIFI®, via near field communication, i.e., Bluetooth® or 4G/5G LTE.

The wireless communication interface 413 is configured to communicate over-the air (OTA) with two functionalities:
1. Upload the signals to the cloud server in the mode of remote communication, and then sent to the mobile APP, and
2. Update the control and driver system and AI signal processing algorithms.

The power management module 411 is responsible for managing the power supply of the entire system, including all voltage conversion and battery charge and discharge management.

The debugger module 407 is connectable to a PC via a USB-type C to update the control and driver system and AI signal processing algorithms and debug the system errors.

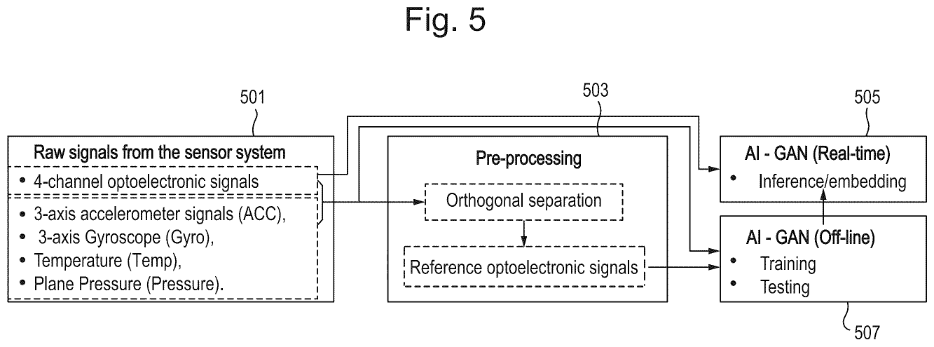

FIG. 5 shows a functional schematic view of an example method of processing signals obtained from an optoelectronic sensor such as via the opto-physiological sensor system of FIGS. 1 to 3 or the opto-physiological sensor system of FIG. 4.

At step 501, raw signals are captured by the multi-wavelength illumination optoelectronic sensor (optoelectronic sensor or photodiode). All raw signals are normalized from 0 to 1. The raw signals may then be pre-processed 503 (for example via orthogonal separation, as is described in more detail below).

The raw signals are then fed into an offline AI 507 and a real-time AI 505 in parallel. The real-time AI 505 is a Generative Adversarial Network (GAN) framework that consists of two generators and one discriminator. Generator1 (G1) aims to learn a noise-resistant mapping from input noisy optoelectronic signals to reference signals. Generator2 (G2) aims to learn the noise distribution captured from the 3-axis accelerometer, the 3-axis gyroscope, the temperature, and the pressure sensors. These two generators, i.e., G1 and G2, are both shared the discriminator (D).

The offline AI 507 is trained as follows:

Training G2 using the reference optoelectronic signals captured from the 3-axis accelerometer, the 3-axis gyroscope, the temperature sensors, and the plane pressure sensors to generate synthetic noisy optoelectronic signals through the loss function L_{G2}.

$${L}_{i n p u t} = {‖ G 2 ( x ) - {p}_{i n p u t} ‖}_{1} \qquad [ Eq . 18 ]$$

where ∥·∥₁ represents the L₁ norm.

$${L}_{r} = 1 - C o r ( G 2 ( x ) , {p}_{i n p u t} ) \qquad [ Eq . 19 ]$$

$$Cor = \frac{{\int }_{0}^{{T}_{M}} G ( x ( t ) ) {p}_{r e f ( t )} d t}{{\int }_{0}^{{T}_{M}} {G ( x ( t ) )}^{2} d t {\int }_{0}^{{T}_{M}} {p}_{ref ( t )}^{2} dt}$$

where

$${L}_{G 2} = \frac{1}{N} {\sum }_{j = 1}^{N} log ( 1 - D 2 ( {p}_{i n p u t} ( j ) ) ) + λ {L}_{i n p u t} ( j ) + β {L}_{r} ( j ) \qquad [ Eq . 20 ]$$

where λ=100, β=100.

Where x is the input of G2 that is contained the reference optoelectronic signals, 3-axis acceleration, 3-axis gyroscope, temperature and pressure signals, and pᵢₙₚᵤₜ is the input noisy optoelectronic signals.

Training G1 using the noisy optoelectronic signals and the synthetic noisy optoelectronic signals according to the loss function L_{G1}.

$${L}_{time \_ 1} = {‖ G 1 ( x ) - {p}_{ref} ‖}_{1} \qquad [ Eq . 21 ]$$

where ∥·λ₁ represents the L1 norm.

$${L}_{fre \_ 1} = {‖ FFT ( G 1 ( x ) ) - FFT ( {p}_{ref} ) ‖}_{1} . \qquad [ Eq . 22 ]$$

$${L}_{G 1\_ 1} = log ( D ( G 1 ( x ) ; x ) ) + λ {L}_{time \_ 1} + β {L}_{fre \_ 1} \qquad [ Eq . 23 ]$$

where λ=100, β=100.

$${L}_{time \_ 2} = {‖ G 1 ( G 2 ( x ) ) - {p}_{ref} ‖}_{1} , \qquad [ Eq . 24 ]$$

where ∥·∥₁ represents the L1 norm.

$${L}_{fre \_ 2} = {‖ FFT ( G 1 ( G 2 ( x ) ) ) - FFT ( {p}_{ref} ) ‖}_{1} \qquad [ Eq . 25 ]$$

$${L}_{G 1 \_ 2} = log ( D ( G 1 ( G 2 ( x ) ) ; G 2 ( x ) ) ) + λ {L}_{time \_ 2} + β {L}_{fre \_ 2} , \qquad [ Eq . 26 ]$$

where λ=100, β=100.

Where x-input noisy PPG signals; G2(x)-input synthetic noise PPG signals Thus,

$${L}_{G 1} = 0.5 {L}_{G 1 \_ 1} + 0.5 {L}_{G 1 \_ 2} \qquad [ Eq . 27 ]$$

Training G1 and G2 by maximizing L_{D} with all generators fixed.

$${max}_{D 1} {min}_{G 1 , G 2} {L}_{D 1} = log ( D 1 ( {p}_{ref} ; x ) ) + log ( 1 - D 1 ( G 1 ( x ) ; x ) ) + log ( D 1 ( {p}_{ref} ; G 2 ( x ) ) ) + log ( 1 - D 1 ( G 1 ( G 2 ( x ) ) ; G 2 ( x ) ) ) \qquad [ Eq . 28 ]$$

where x− input noisy optoelectronic signals. G2(x)-input synthetic noise noisy optoelectronic signals.

The procedure repeats the above training strategy to test the model until the stability establishment (Verification).

Real-time dataset: A real-time dataset is established once the multi-wavelength illumination optoelectronic sensor is applied on subject(s) for a test. In compliance with clinical monitoring standards, a sampling rate of >1 kHz for each channel from the optoelectronic sensor is accounted during cycling, walking, jogging and running or other routine physical intensive activities.

Configuration setting: The GAN is implemented in TensorFlow and trained on NVIDIA RTX 3080Ti, or equivalents All CNNs are trained for 200 epochs, using random initialization via Adam optimizer with 0.5 and 0.99, weight decay of 0, the learning rate of 0.0001, batch size of 8 and without any decay strategy for learning rate.

The real-time AI 505 operates as follows: (Inference/Embedding on MCU)

Inference procedure: G1 can easily port into the embedded sensor system.

When the off-line GAN 507 (Training and testing) via the cloud computing programme/algorithm, completes the training procedure, the training weights are generated with the real-time dataset provided by the sensor system. The training weights are shared with the inference procedure of the GAN model. Thus, the inference can be established into the embedding MCU 405, and is used to generate target optoelectronic signals via the input noisy optoelectronic signals.

It is therefore understood that the micro control unit 405 is trained by obtaining a pulsatile signal providing an indication of at least one physiological property of a subject, obtaining an indication of at least one physical variable as a function of time from the wearable device, sending data representative of the pulsatile signal and the indication of the physical variable via the wireless interface 413 to a remote computing platform, i.e., workstation or a computing cloud as shown in FIG. 4, training, at the remote computing platform, a machine learning model based on the received data, and sending the coefficients/parameters, after the training, from the remote computing platform to an embedded machine learning model in the wearable device.

It is therefore also understood that the micro control unit 405 may be trained by obtaining a pulsatile signal providing an indication of at least one physiological property of a subject, obtaining an indication of at least one physical variable as a function of time from the wearable device, processing the pulsatile signal and the indication of the at least one physical variable using a machine learning model to provide an output indicative of the physiological property, sending data representative of the pulsatile signal and the indication of the physical variable via a wireless interface to a remote computing platform, training, at the remote computing platform, an improved machine learning model based on the received data; sending the improved, trained, machine learning model to the wearable device; and using the improved, trained, machine learning model to process the pulsatile signal and the indication of the at least one physical variable using a machine learning model to provide an output indicative of the physiological property.

For intense activities the signals are pre-processed 503 via orthogonal separation as described below. It is understood that in some examples the micro control unit 405 is configured to determine whether the wearer/subject is undertaking intense activities for example based on data from the sensor module 401. For example, the micro control unit 405 may determine that the wearer/subject is undertaking intense activities in the event that the sensor module indicates a physical variable (e.g., acceleration) outside of a selected range or above a selected threshold level.

The light intensity I at the photodiode consists of 1) a large DC contribution I_{DC} from light scattered by local tissue, and 2) a much smaller AC component I_{AC}(t) which includes the pulsatile signal (or pulsatile wave or pulsatile waveform) together with other time-dependent effects. In the case of intense activity (IA), a significant contribution to the measured AC signal can be attributed to motion artefacts. Although the motion contribution to the overall measured signal I is small, its presence can be clearly identified in the similarly small AC signal I_{AC}(t). In the case of high intensity activity, the motion artefacts can come to dominate the small pulsatile signal, masking its properties and making biomarker assessment particularly difficult.

The motion, as physical factors, e.g., acceleration and balance, is captured by the 3-axis accelerometer, the 3-axis gyroscope, and the contact force sensor during the measurement period T_{M}, affects the light-tissue interaction in terms of AC component I_{AC}(t).

The motion factors affecting the AC signal from the 3-axis accelerometer, 3-axis gyroscope, contact force, touch skin temperature are taken account into the raw signals captured by the optoelectronic sensor system and expressed as:

$${s}_{o r t h o g} ( t ) = s ( t ) - {s}_{m} ( t ) \qquad [ Eq . 29 ]$$

$${s}_{o r t h o g} ( t ) = s ( t ) - [ {s}_{a} ( t ) + {s}_{b} ( t ) + {s}_{p} ( t ) + {s}_{T} ( t ) ]$$

where sₒᵣₜₕₒ(t) is the orthogonal signal with respect to time(t), s(t) is the generated signal, and sₘ(t) is the signal composed of the motion factors, including the acceleration component sₐ(t), pressure component sₚ(t), temperature component sₜ(t), and balance component s_{b}(t).

The detail of how this motion, as measured by the 3-axis accelerometer, 3-axis gyroscope in the system, affects the light-tissue interaction, and thus I_{AC}(t), depends on the location and the method of attachment to the tissue body. In any case of repetitive exercise motion, an alternating series of tissue compressions and expansions is created which modulates the contact force as detected by the contact plane pressure sensor 123. For regular (periodic) exercise, AC light signal is impacted in two ways. Firstly, the strain in the tissue region d(t) is driven by the periodic motion and, in a linear-elastic model, each driving frequency, x(t)=sin(ωt), produces a delayed response d(t)=A(ω)sin(ωt+φ(ω)). Secondly, in compression, the strain produces a back-force on the peripheral blood flow; increasing the local effective vascular resistance; reducing blood volume and thus also the amplitude of pulsatile signal. The strain also affects tissue absorption and scattering properties through its impact on the concentration C of absorbing/scattering centres.

Thus, for example, the parameter λₐ(n) which defines the absorption per unit path length for tissue type n (dermis, epidermis, etc.), can be written in terms of the extinction coefficient ϵ and the absorber concentration C for that tissue type in the form μₐ(n)=ϵ(n)×C(n).

Considering the detected light intensity I (once band-pass filtered) as approximately a Taylor Series

$$I ( \underline{osc} = [ pulse , a , p , T , b ] ) = I ( {\underline{osc}}_{0} ) + ( \underline{osc} - {\underline{osc}}_{0} ) \cdot ∇ I + … = {I}_{D C} + {s}_{o r t h o g} ( t ) + {s}_{a} ( t ) + {s}_{p} ( t ) + {s}_{b} ( t ) + {s}_{T} ( t )$$

to first order in small time-dependent (oscillating (osc)) terms, I_{AC}(t) is a linear combination of the pulsatile signal and motion terms which consists of a number of delayed responses to forcing term frequencies and which, equivalently, adds forcing frequencies into the I_{AC} spectrum. Such frequencies are then removed to recover the pulsatile signal. For regular exercise, it is sufficient to include the dominant forcing frequencies measured by the accelerometers.

In Orthogonal Separation, these dominant frequencies are removed by separating the space F of ‘functions defined over some time interval T_{M}’ (generally this time interval is an 8-10 second window, advancing one second at a time), into a subspace A spanned by aₖ(t), k=1 . . . 6, the 3-axis accelerometer readings (and their integrated velocities), and the complementary orthogonal subspace P. The Orthogonal Separation method obtains the pulsatile signal as the projection of the measured AC signal s(t) onto the orthogonal subspace P; a construction equivalent to minimising the integral

$$f ( N ) = {\int }_{0}^{{T}_{M}} {( s ( t ) - {\sum }_{i = 1}^{6} {N}_{k} {a}_{k} ( t ) )}^{2} d t \qquad [ Eq . 30 ]$$

with respect to variations in the Nₖ. As is common, all signals must be filtered first to remove any: DC component; low frequency baseline drift; and high-frequency noise, but it is crucial that the band-pass filter used prior to the orthogonal separation is of zero-phase.

This process may be done very rapidly (<<1 sec), making it well-suited to use in a real-time physiological monitoring system. When the true pulsatile signal and the accelerometer signals occupy different regions of the frequency domain, this method is exact. While if the signals overlap in frequency space, the method still works very well for extracting biomedical markers since peaks in the forcing frequency are significantly sharper than those in the pulsatile signal (largely due to frequency spreading caused by the presence of the surrounding soft tissue).

The impact of Orthogonal Separation in that case is to reduce the height of the broad pulsatile frequency peaks, but not to remove them. Even in this rare case, there is sufficient signal integrity to obtain Heart (HR), Respiration Rates (RR), Oxygen Saturation (SpO₂) levels, pulse transmitted time (PTT), and pulse wave velocity (PWV), and other biomarkers.

The four wavelengths provided by the device advantageously allow all such biomarkers to be obtained at each wavelength, increasing confidence in the results and further noise reduction through Independent Component Analysis (ICA). The four wavelengths used in the optoelectronic sensor advantageously allow for additional quality control through a piece of analysis which is also an important part of the assessment of Oxygen Saturation levels (SpO₂). SpO₂ levels are often obtained using the Ratio-of-Ratios method and it has been observed by many authors that the ratio σ(λ₁, λ₂) of AC/DC value at wavelength λ₁ to the AC/DC value at wavelength λ₂ is a reasonable proxy, simply related to the SpO₂ value.

SpO₂ validation with gold standard data often fits well to some low degree polynomial; typically, linear or quadratic, in σ(λ₁, λ₂). The optoelectronic sensor measurements displayed as a plot of σ(λ₁: 650 nm, λ₂:910 nm) values against σ(λ₁: 525 nm, λ₂: 595 nm) values show a very good linear relationship, meaning that it is possible to define the proxy parameter Ĥ (independent of which pair of wavelengths is used)

$$\hat{H} ( t ) = A ( {λ}_{1} , {λ}_{2} ) σ ( {λ}_{1} , {λ}_{2} ) + B ( {λ}_{1} , {λ}_{2} ) = A ( {λ}_{3} , {λ}_{4} ) σ ( {λ}_{3} , {λ}_{4} ) + B ( {λ}_{3} , {λ}_{4} ) \qquad Eq . [ 31 ]$$

On the one hand this supports the general assumption that there is a parameter Ĥ(t) which is a proxy biomarker and (assuming, say, a quadratic fit for SpO₂), gives SpO₂=aĤ²+bĤ+c, for some a, b, c. Additionally, though, tracking the linearity of the σ−σ fit provides a useful check on signal integrity.

This is particularly important in cases where the SpO₂ methodology works least well, such as in low temperature conditions as monitored by the device's temperature sensor on for darker skin tones.

It is understood that obtaining such a biomarker does not require a wearable device but may instead be determined for example through the use of a camera or other means operable to receive and distinguish light of the distinct wavelengths.

In some examples, the micro control unit 405 is configured to determine the dominant or dominant forms of noise, for example based on physical variables (for example as obtained via the sensor module 401) and apply a selected model based on the dominant or dominant forms of noise to remove the noise in the pulsatile signal. For example, if the dominant form of noise is due to temperature, the micro control unit 405 may be configured to apply a model based on temperature. In some examples a model may only be applied if the noise reaches a selected threshold level of noise—for example if the noise from a determined source is above a selected threshold level. Applying a model only to the dominant or dominant forms of noise, rather than always applying all models, may advantageously improve the real-time processing of the signals. This in turn may advantageously improve real-time output of physiological parameters.

As noted above, in some examples the micro control unit 405 is also configured not to obtain data if a parameter is outside a selected threshold range. For example, if the temperature reading is outside of a selected range, or if the contact pressure is outside of a selected range, the micro control unit 405 is configured not to obtain any data and/or send any data via the wireless communication interface 413.

As noted above, the opto-physiological sensor and method of monitoring may be well suited to accommodate different physiological variables, such as tissue types, ages, body masses tissue locations, present of tattoos and skin tone/pigmentation, with an auto-attenuation function to automatically select the illumination intensity suited for the optoelectronic sensor operation.

The performance of the opto-physiological monitoring system has been tested with six healthy volunteers. The system performance is evaluated for the tissue type, tissue location and skin pigmentation. The Analog Front End driving multiplexed LEDs (four wavelengths or more: 525 nm green, 595 nm orange, 650 nm red, and 910 nm infra-red), provides high-resolution pulsatile waveforms (<1 ms) to capture variations in light trans-illuminating tissues. These wavelengths used are merely example wavelengths, and other wavelengths may be used, for example in the range of 465 to 1450 nm. For example, the wavelengths used may be based on the individual's physiological variables (such as tissue types, ages, body masses tissue locations, present of tattoos and skin tone/pigmentation). For example, different wavelengths may be used for a neonate with light skin tone compared to an adult with dark skin tone.

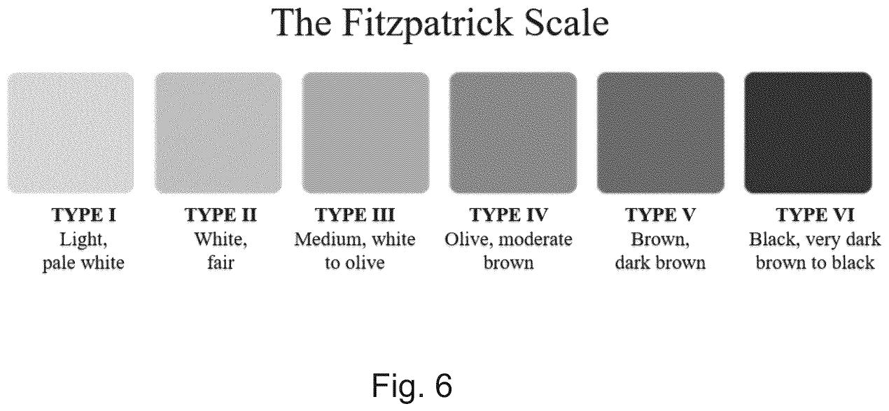

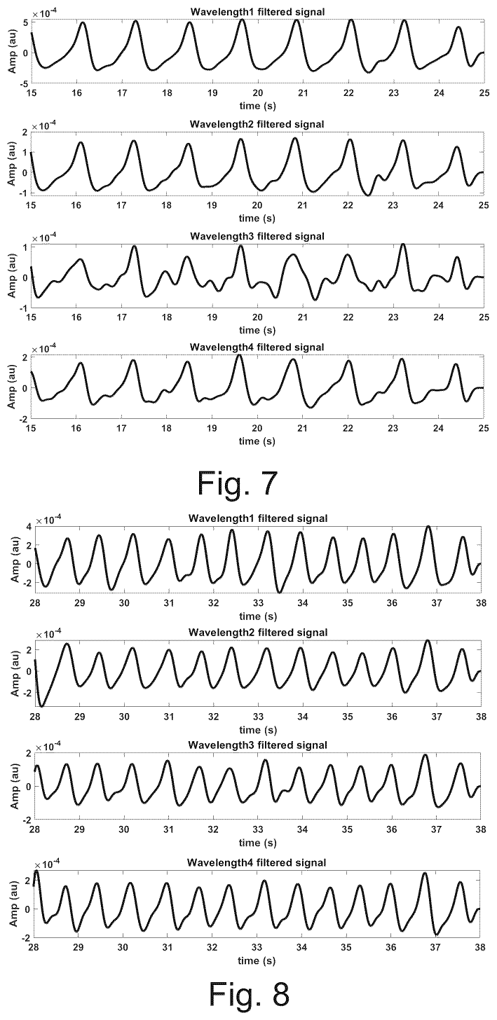

FIG. 6 shows the Fitzpatrick scale (Source: Hypoxia Lab, “Protocol for skin color assessment for pulse oximeter performance studies”. August 2022. https://openoximetry.org/study-protocols). The opto-physiological monitoring system is tested for skin types I-VI (skin types were determined using the Fitzpatrick scale in FIG. 6). The results displayed in FIGS. 7 to 12 are for six healthy volunteers: Four males (skin types I, III, VI, VI) and two females (skin types II, V). For each subject, measurements were collected at room temperature (20±3° C.), at rest, for a duration of one minute.

FIGS. 6 to 12 show pulsatile waveforms captured from four wavelength illuminations by the opto-physiological sensor of embodiments of the disclosure, such as the opto-physiological sensors of FIGS. 1 to 3.

FIG. 6 shows pulsatile waveforms for a subject with skin type I (Fitzpatrick scale) collected from the back of the left wrist using the opto-physiological monitoring system.

FIG. 7 shows pulsatile waveforms for a subject with skin type II (Fitzpatrick scale) collected from the back of the left wrist using the opto-physiological monitoring system.

FIG. 8 shows pulsatile waveforms for a subject with skin type III (Fitzpatrick scale) collected from the back of the left wrist using the opto-physiological monitoring system.

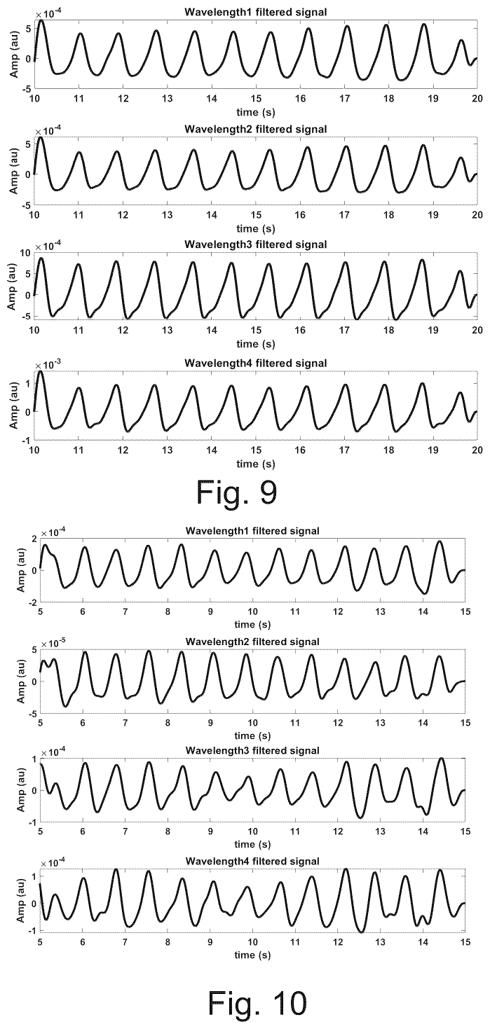

FIG. 9 shows pulsatile waveforms for a subject with skin type IV (Fitzpatrick scale) collected from the back of the left wrist using the opto-physiological monitoring system.

FIG. 10 shows pulsatile waveforms for a subject with skin type V (Fitzpatrick scale) collected from the back of the left wrist using the opto-physiological monitoring system.

FIG. 11 shows pulsatile waveforms for a subject with skin type VI (Fitzpatrick scale) collected from the back of the left wrist using the opto-physiological monitoring system.

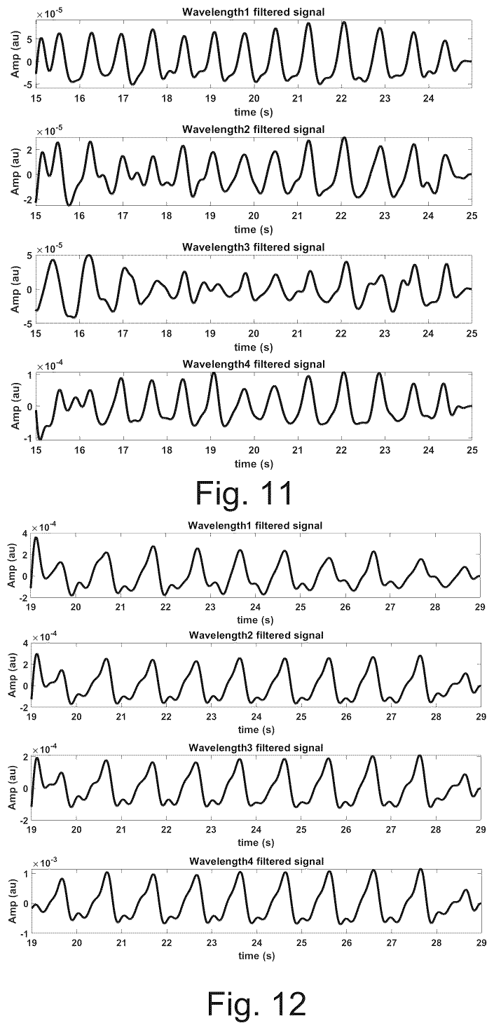

FIG. 12 shows pulsatile waveforms for a subject with skin type V (Fitzpatrick scale) collected from the back of the left wrist using the opto-physiological monitoring system.

SpO₂ comparative results for the opto-physiological monitoring system as described above with respect to FIG. 1, compared with Masimo's LNCS® TFA-1™ Disposable Transflectance Sensor, and NONIN 8000R Reflectance SpO₂ Sensor, and are displayed in Table 1 below. The measurement site for this test is the forehead. The skin types were determined using the Fitzpatrick scale. Healthy volunteers with skin types II, III, V, and VI were tested. For each subject, measurements were collected at room temperature (20±3° C.), at rest, for a duration of one minute.

TABLE 1
Comparative SpO2 results for the opto-physiological monitoring
system, Masimo's LNCS ® TFA-1 ™ Disposable
Transflectance Sensor, and NONIN 8000R Reflectance SpO2 Sensor.

| | | Masimo's LNCS | NONIN 8000R |
|---|---|---|---|
| | Opto-physiological | TFA-1 Disposable | Reflectance SpO2 |
| | monitoring system | Transflectance | Sensor (index finger, |
| Skin Type | (forehead) | Sensor (forehead) | left hand) |
| Skin Type II | 99% | 98% | 96% |
| Skin Type III | 98% | 98% | 99% |
| Skin Type V | 97% | 95% | 95% |
| Skin Type VI | 99% | Invalid (Red channel: | 99% |
| | | no valid signal) | |

Perfusion Index (PI) comparative results for the opto-physiological monitoring system as described above with respect to FIG. 1 compared against NONIN 8000R Reflectance SpO₂ Sensor and Masimo's LNCS® TFA-1™ Disposable Transflectance Sensor are displayed in Table 2. The measurement sites for this test are the wrist, the index finger, and the forehead. The skin types were determined using the Fitzpatrick scale. Healthy volunteers with skin types II, III, V, and VI were tested. For each subject, measurements were collected at room temperature (20±3° C.), at rest, for a duration of one minute. The results displayed in FIG. 8 are for a healthy volunteer with skin type VI.

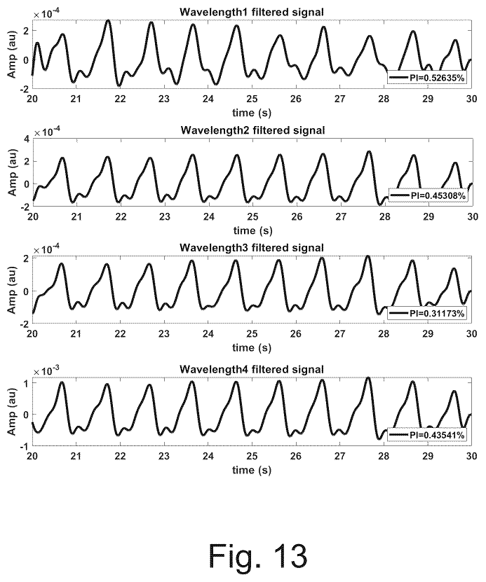

FIG. 13 shows pulsative waveforms of male subject skin type VI (Fitzpatrick scale) with 4 wavelength opto-physiological monitoring. Subject at rest, measurement site: back of the left wrist. Room temperature: 20° C. (±2).

TABLE 2
PI(%) average results for the opto-physiological sensor of the
present disclosure for four subjects (skin types II, III, V,
VI) and two commercial clinical grade pulse oximetry probes.

| | | | NONIN 8000R | | Masimo's LNCS ® | |
|---|---|---|---|---|---|---|
| | Opto- | | Reflectance | | TFA-1 ™ Disposable | |
| | physiological | | SpO2 Sensor | | Transflectance | |
| Skin | Sensor | | | Index | Sensor | |
| Type | Wrist | Forehead | Wrist | finger | Wrist | Forehead |
| II | 0.22 | 0.41 | Invalid | 3.38 | 0.42 | 0.32 |
| III | 0.40 | 0.66 | 0.08 | 3.48 | 0.14 | 0.24 |
| V | 0.23 | 1.10 | 0.12 | 0.34 | 0.58 | 0.20 |
| VI | 0.43 | 0.34 | 0.24 | 2.20 | 0.18 | 0.81 |
| | | | | | (Red channel | (Red channel |
| | | | | | invalid) | invalid) |

The embodiments shown in the Figures are merely exemplary, and include features which may be generalised, removed or replaced as described herein and as set out in the claims. In the context of the present disclosure other examples and variations of the apparatus and methods described herein will be apparent to a person of skill in the art.

1. A method of monitoring a subject with an opto-physiological sensor system, the opto-physiological sensor system comprising at least one sensor, the method comprising:
obtaining a model of the optical properties of at least one body tissue type to be monitored, wherein the model of the optical properties comprises a definition of static and dynamic components of transmitted optical power and a definition of a source-detector separation related to a normalised path length for an illumination source of the opto-physiological sensor;
obtaining an indication of at least one physiological property of the subject from a wearable device comprising the at least one sensor worn by the subject;
obtaining an indication of at least one physical variable from the wearable device comprising the at least one sensor worn by the subject;
determining, using the model, how the at least one physical variable affects the at least one physiological property; and
determining a corrected value for the physiological property based on the determination of how the at least one physical variable affects the at least one physiological property.

2. The method of claim 1 further comprising determining measurements of accurate physiological parameters using the corrected value for the physiological property, the physiological parameters comprising at least one of: heart rate (HR), perfusion index (PI), oxygen saturation (SpO₂%), respiration rate (RR), blood pressure (BP), pulse rate variability (PRV), pulse transmitted time (PTT), and pulse wave velocity (PWV).

3. The method of monitoring a subject with an opto-physiological sensor of claim 1 wherein the at least one physical variable comprises at least one of: contact pressure; contact temperature; acceleration; angular velocity; and absolute orientation.

4. The method according to claim 1 wherein the model comprises defining infinitesimal optical power dP that is transmitted through to point (x, y) on a surface of the body tissue type as per Equation 1:
$$ρ ( λ , {l}^{′} ( {x}^{′} , {y}^{′} ) ) : dP ( λ , x , y ) = \int \int {I}_{0} ( λ , {x}^{′} , {y}^{′} ) \times {e}^{- ρ ( λ , {l}^{′} ( {x}^{′} , {y}^{′} ) )} {dx}^{′} {dy}^{′} \qquad [ Eq . 1 ]$$
wherein Iₒ (λ, x, y) represents the total light that enters across the entire surface of the body tissue type and is subject to exponential decay, which is a function of the optical density ρ(λ) of the body tissue type;
wherein a source-detector separation is defined in terms of an illumination source at point (xₛ, yₛ) and an arbitrary detector at point (x, y) on the surface of the body tissue type, such that l′(x, y)=\sqrt{(x−xₛ)²+(y−yₛ)²}; and
wherein for a detector of a finite rectangular area on the surface of the body tissue type defined by vectors x and y, spanning from x₋ to x₊ and from y₋ to y₊, the optical power received by the detector is as per Equation 2:
$$P ( λ , x , y ) = {\int }_{{x}_{-}}^{{x}_{+}} {\int }_{{y}_{-}}^{{y}_{+}} d P ( λ , {x}_{d} , {y}_{d} ) d {x}_{d} d {y}_{d} . \qquad [ Eq . 2 ]$$

5. The method according to claim 1 wherein the model comprises defining an optical response of a dynamic and multi-layered body tissue type in terms of its dynamic optical density ρ(λ, l′, t), using a normalised physiological pulse function ψ(t), and absorption, scattering and pulsatility coefficients μₐᵢ(λ), μₛᵢ(λ) and μₚᵢ(λ) respectively, where a layer number i ranges from 1 to N as per Equation 3:
$$ρ ( λ , {l}^{′} , t ) = {\sum }_{i = 1}^{N} ( {μ}_{a i} ( λ ) \times L ( {μ}_{s i} ( λ ) , {l}^{′} ) \times ( 1 + {μ}_{p i} ( λ ) \times ψ ( t ) ) ) . \qquad [ Eq . 3 ]$$

6. The method according to claim 4 wherein the model comprises separation of static ρ̅(λ, l′) and dynamic \tilde{ρ}(λ, l′, t) components of Equation 4 as per Equations 5 to 6:
$$ρ ( λ , {l}^{′} , t ) = \overline{ρ} ( λ , {l}^{′} ) + \tilde{ρ} ( λ , {l}^{′} , t ) \qquad [ Eq . 4 ]$$
$$\overline{ρ} ( λ , {l}^{′} ) = {\sum }_{i = 1}^{N} ( {μ}_{a i} ( λ ) \times L ( {μ}_{s i} ( λ ) , {l}^{′} ) ) \qquad [ Eq . 5 ]$$
$$\tilde{ρ} ( λ , {l}^{′} , t ) = {\sum }_{i = 1}^{N} ( {μ}_{a i} ( λ ) \times L ( {μ}_{s i} ( λ ) , {l}^{′} ) \times {μ}_{p i} ( λ ) \times ψ ( t ) ) . \qquad [ Eq . 6 ]$$

7. The method according to claim 6 wherein the model comprises using a sum rule of integration on Equations 1 and 2 to define static P̅(λ, x, y) and dynamic \tilde{P}(λ, x, y, t) components of transmitted optical power for a rectangular detector defined by vectors x and y as per Equations 7, 8 and 9:
$$P ( λ , x , y , t ) = \overline{P} ( λ , x , y ) + \tilde{P} ( λ , x , y , t ) \qquad [ Eq . 7 ]$$
$$\overline{P} ( λ , x , y ) = {\int }_{{x}_{-}}^{{x}_{+}} {\int }_{{y}_{-}}^{{y}_{+}} ( \int \int {I}_{0} ( λ , {x}^{′} , {y}^{′} ) \times {e}^{- \overline{ρ} ( λ , {l}^{′} ( {x}^{′} , {y}^{′} ) )} {dx}^{′} {dy}^{′} ) d {x}_{d} d {y}_{d} \qquad [ Eq . 8 ]$$
$$\tilde{P} ( λ , x , y , t ) = {\int }_{{x}_{-}}^{{x}_{+}} {\int }_{{y}_{-}}^{{y}_{+}} ( \int \int {I}_{0} ( λ , {x}^{′} , {y}^{′} ) \times {e}^{- \tilde{ρ} ( λ , {l}^{′} ( {x}^{′} , {y}^{′} ) , t )} {dx}^{′} {dy}^{′} ) {dx}_{d} {dy}_{d} . \qquad [ Eq . 9 ]$$

8. The method according to claim 7 wherein the model comprises defining an optimum source-detector separation l′(λ) for an illumination source at wavelength λ as per Equation 10:
$${l}^{′} ( λ ) = {max}_{( x , y )} ( \vert {min}_{t} ( \tilde{P} ( λ , x - x , y - y , t ) ) \vert / \overline{P} ( λ , x - x , y - y ) ) . \qquad [ Eq . 10 ]$$

9. The method according to claim 1 wherein the model comprises assuming cylindrical symmetry and optical homogeneity of the body tissue type to be monitored such that an optimum source-detector separation l′(λ) is expressed as a circle centred on the position of the illumination source (xₛ, yₛ), or conversely centred on the position of the detector (x_{d}, y_{d}), as per Equation 11:
$${l}^{′} ( λ ) = \sqrt{{( x - {x}_{s} )}^{2} + {( y - {y}_{s} )}^{2}} = \sqrt{{( x - {x}_{d} )}^{2} + {( y - {y}_{d} )}^{2}} . \qquad [ Eq . 11 ]$$

10. The method according to claim 1 wherein the at least one physical variable comprises at least one of acceleration, angular velocity and absolute orientation, and wherein determining, using the model, how the at least one physical variable affects the at least one physiological property comprises modelling the pumping action of the heart to determine a volumetric blood flow rate.

11. The method of claim 10 wherein modelling the pumping action of the heart comprises determining a pressure gradient in the form of Equation 12
$$\frac{\partial p}{\partial z} = {A}_{0} + {A}_{1} cos {ω}_{p} t \qquad [ Eq . 12 ]$$
where A₀ is the constant component of the blood pressure gradient from the heart, A₁ is the amplitude of the fluctuating component and ωₚ=2λfₚ where fₚ is the pulse frequency, and determining the volumetric blood flow rate Q via equations 13 and 14
$$ρ \frac{\partial {u}_{z}}{\partial t} = ρ {a}_{0} cos ( {ω}_{0} t + ∅ ) + {A}_{0} + {A}_{1} cos {ω}_{p} t + {μ}_{f} ( \frac{{\partial }^{2} {u}_{z}}{\partial {r}^{2}} + \frac{1}{r} \frac{\partial {u}_{z}}{\partial r} ) \qquad [ Eq . 13 ]$$
$$Q = 2 π {\int }_{0}^{R} r \overline{{u}_{z}} dr \qquad [ Eq . 14 ]$$
where ρ and μ_{f} are the density and viscosity respectively of the blood flowing through the blood vessels, and u_{z} is the velocity of the blood flow in the axial direction, and where the z-axis is taken along the axis of the arterial blood segment, and r is taken along the radial direction as the combination of acceleration, angular velocity; and absolute orientation hat may be obtained when obtaining the indication of the at least one physical variable from the wearable device.

12. The method according to claim 1 wherein the at least one physical variable comprises temperature, and wherein determining, using the model, how the at least one physical variable affects the at least one physiological property comprises modelling the density of a specific tissue type as a function of wavelength of the illumination source of the opto-physiological sensor and temperature.

13. The method of claim 12 wherein modelling the density of a specific tissue type as a function of wavelength of the illumination source of the opto-physiological sensor and temperature comprises modelling tissue temperature as a result of blood perfusion as defined by thermoregulation as expressed per equation 15, that reflects the changes from tissue composition, skin thickness, surface area, tissue volume, and ambient temperature in presence of live tissue nature or body tissue surroundings:
$$ρ ( λ , t ) {C}_{p} V \frac{dT}{dt} = {h}_{air} A ( T - {T}_{air} ) + {ρ}_{b} {C}_{{p}_{b}} {ω}_{b} ( t ) ( {T}_{A} - T ) \qquad [ Eq . 15 ]$$
where ρ(λ,t) indicates the density of a specific tissue type, Cₚ is the tissue specific heat, V is the specific tissue volume, and A is the tissue surface area, such that A=πD²/2, and wherein V and A are dependent on the average tissue diameter D=2R, where R is the average radius of the tissue surface area A, and wherein the density and specific heat of blood are denoted by ρ_{b} and C_{pb} respectively, whereas ω_{b} is the volumetric blood flow, and wherein T_{A} is the arterial temperature and hₐᵢᵣ is the heat transfer coefficient at the skin surface as dominated by the environmental heat exchange, and Tair represents the temperature of the surrounding.

14. The method of claim 13 wherein modelling the density of a specific tissue type as a function of wavelength of the illumination source of the opto-physiological sensor and temperature comprises modelling tissue temperature as a result of blood perfusion as defined by thermoregulation for multi-layers with multi-wavelength illuminations as per equation 16:
$${ρ ( λ , t )}_{N} {C}_{p N} \frac{d {T}_{N}}{d t} = {k}_{N} {∇}^{2} {T}_{N} + {ρ}_{b} {C}_{{p}_{b}} {ω}_{b N} ( t ) ( {T}_{A} - {T}_{N} ) + {q}_{m N} \qquad [ Eq . 16 ]$$
where k_{N}, ρ_{N}, C_{pN}, ω_{bN}, and q_{mN} represent the thermal conductivity, density, specific heat, blood perfusion and metabolic heat generation of the respective tissue layer (N), ρ_{b} is the blood density, whereas C_{pb} represents the specific heat of blood and T_{A} is the body temperature and is treated as a constant.

15. The method according to claim 1 wherein the at least one physical variable comprises contact pressure of the opto-physiological sensor with the subject's skin, and wherein determining, using the model, how the at least one physical variable affects the at least one physiological property comprises modelling a change in the optical density of a specific tissue type based on a change in the optical path length.

16. The method of claim 15 wherein determining, using the model, how the at least one physical variable affects the at least one physiological property comprises modelling the optical density of a specific tissue type as a function of wavelength and temperature as being proportional to the contact force when the contact force is within a selected range, and modelling the optical density of a specific tissue type as a function of wavelength and temperature as being proportional to the inverse of the contact force when the contact force is outside of the selected range.

17. The method of claim 15 comprising modelling the changes in the optical path length due to increased or decreased contact force as inducing a change in the optical density ρ(Δ, t) in terms of its AC and DC components given by \tilde{ρ} and ρ̅ respectively, such that
$$\{ \begin{matrix}ρ ( λ , t ) ∝ {F}_{c} , & 0.15 < {F}_{c} < 1.5 \\ ρ ( λ , t ) ∝ \frac{1}{{F}_{c}} , & otherwise\end{matrix} . \qquad [ Eq . 17 ]$$

18.-56. (canceled)

57. A computer readable non-transitory storage medium comprising a program for a computer configured to cause a processor to perform the method of claim 1.
